(19) United States
(12) Patent Application Publication
(10) Pub. No.: US 2016/0355539 A1
(43) Pub. Date: Dec. 8, 2016

(54) SELECTIVE SOLVENT FREE PHOSPHORYLATION

(71) Applicants: ChromaDex, Inc., Irvine, CA (US); The Queen's University of Belfast, Belfast (GB)

(72) Inventors: Marie Eugenie Migaud, Lurgan (GB); Philip Redpath, Portadown (GB); Kerri Crossey, Magherafelt (GB); Richard Cunningham, Portadown (GB); Troy Rhonemus, Mission Viejo, CA (US); Sylesh Venkataraman, Frisco, TX (US)

(73) Assignees: ChromaDex, Inc., Irvine, CA (US); The Queen's University of Belfast, Belfast (GB)

(21) Appl. No.: 15/172,662

(22) Filed: Jun. 3, 2016

Related U.S. Application Data
(60) Provisional application No. 62/171,138, filed on Jun. 4, 2015.

(51) Int. Cl. C07H 19/048; C07F 9/58; C07H 19/20; C07F 9/6558

(52) U.S. Cl. CPC C07H 19/048; C07F 9/65583; C07F 9/585; C07H 19/20

(57) ABSTRACT

A synthetic process is provided for the preparation of phosphorylated analogs of nicotinamide riboside (“NR”) having the formula (I), or salts thereof, and reduced or modified derivatives thereof, having the formula (II), wherein X⁻, Y¹, Y², Z¹, Z², n, R¹, R², R³, R⁴, R⁵, R⁶, and R⁷ are as defined herein. The present disclosure also relates to the preparation of phosphorylated analogs of nicotinic acid riboside (“NAR”) having the formula (I), or salts thereof, and reduced or modified derivatives thereof, having the formula (II). Generally solvent-free conditions are employed using appropriate mechano-chemical techniques as described.

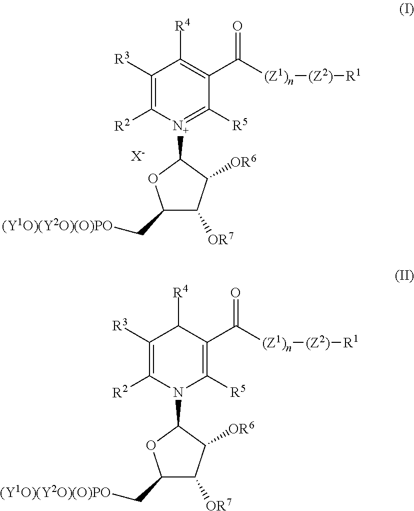

This application claims the benefit of U.S. Provisional Application Ser. No. 62/171,138, filed on Jun. 4, 2015. The disclosure of this prior application is incorporated herein by reference in its entirety for all purposes.

### TECHNICAL FIELD

The present disclosure relates to a synthetic process for the preparation of phosphorylated analogs of nicotinamide riboside (“NR”) or nicotinic acid riboside (“NAR”) and reduced or modified derivatives thereof. The present disclosure also relates to the preparation of phosphorylated analogs of nicotinic acid riboside (“NAR”) and reduced or modified derivatives thereof. The present disclosure also relates to the preparation of monophosphorylated species in an atom-efficient manner under phosphate solvent-free conditions using appropriate mechano-chemical techniques as described.

### BACKGROUND

Despite extensive optimization of solution-based methodologies over many years for nucleotide preparation, difficulties and issues remain in the monophosphorylation of active hydroxyl groups, with respect to low yields and product stability and isolation from polar solvents. The current methodology is also plagued by atom inefficiency due to the high molar ratio of phosphorus reagent compared to nucleoside starting materials.

Synthetically, the preparation of 5′-nucleotides remains time-consuming, atom-inefficient, and costly, due to the need for numerous protection and deprotection steps. In these preparation methods, the chlorodialkylphosphate, tetraalkylpyrophosphate, chlorophosphite, or phosphoramidite reagents required are also expensive starting materials by virtue of their chemical functionalization and chemical instability, and therefore, consequent associated synthetic difficulties.

One known alternative approach to the protection/deprotection method is to use phosphorus oxychloride (P(O)Cl₃) (i.e., Yoshikawa conditions), however there are still drawbacks to this method, as follows. In this method, polar trialkyl phosphate solvents, such as P(O)(OMe)₃, are used in a large excess, which enhances reaction rates while limiting the undesirable reactivity of P(O)Cl₃ as a chlorinating agent. Thus, it is believed that the excess P(O)Cl₃/P(O)(OR)₃ is a better combination for the chemoselective 5′-O-phosphorylation of unprotected ribosides. However, the use of trialkyl phosphate solvents, such as P(O)(OMe)₃, precludes their implementation for the preparation of materials for eventual human use, as this class of solvent is highly toxic (known carcinogen, non-GRAS approved) and is difficult to remove from the final polar products. See M. Yoshikawa et al., Studies of Phosphorylation. III. Selective Phosphorylation of Unprotected Nucleosides, 42 BULL. CHEM. SOC. JAPAN 3505 (1969); Jaemoon Lee et al., A chemical synthesis of nicotinamide adenine dinucleotide (NAD—), CHEM. COMMUN. 729 (1999); incorporated by reference herein in their entireties.

In another alternative approach, enzymatic hydrolysis of NAD⁺ and NADH is known for the production of nicotinamide mononucleotide (“NMN”) and of its reduced form. See Frank Friedlos & Richard J. Knox, Metabolism of nad(p)h by blood components: Relevance to bioreductively activated prodrugs in a targeted enzyme therapy system, 44 BIOCHEMICAL PHARMACOLOGY 631 (1992), incorporated by reference herein in its entirety.

In view of the above, there is a need for a process that is atom-efficient in terms of reagent equivalency, that bypasses the need for polar solvents, that is versatile in terms of limitations associated with solubility and reagent mixing, and finally that is time- and energy-efficient, to provide an efficient and practical method for the phosphorylation of 5′-ribosides, particularly nicotinamide or nicotinate ribosides, and their respective reduced forms, by chemoselective and solvent-free methods.

### SUMMARY OF THE INVENTION

In accordance with one embodiment, the present disclosure provides a method of phosphorylation of active hydroxyl groups, for application to 5′-phosphonucleoriboside production and to B-vitamins such as vitamins B1, B3, and B6. In an embodiment, the phosphorylation method can be applied for the preparation of mononucleotide conjugates or esters with B-vitamins such as B1, B3, and B6.

In an embodiment, the preparation of phosphorylated analogs of nicotinamide riboside (“NR”) and modified derivatives thereof is provided. The present disclosure also relates to the preparation of phosphorylated analogs of nicotinic acid riboside (“NAR”) and reduced or modified derivatives thereof. Prototype product ribonucleotide compounds include compounds having formula (I), or a salt thereof:

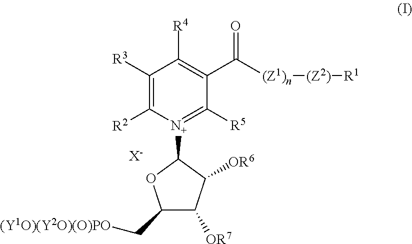

optionally wherein X⁻ as counterion is absent, or when X⁻ is present X⁻ is selected from the group consisting of fluoride, chloride, bromide, iodide, formate, acetate, ascorbate, benzoate, carbonate, citrate, carbamate, formate, gluconate, lactate, methyl bromide, methyl sulfate, nitrate, phosphate, diphosphate, succinate, sulfate, trifluoromethanesulfonate, and trifluoroacetate;

optionally wherein when X⁻ is absent, optionally the counterion is an internal salt;

Y¹ and Y² are independently selected from the group consisting of hydrogen, sodium, potassium, lithium, substituted or unsubstituted (C₁-C₈)alkyl, substituted or unsubstituted (C₁-C₈)cycloalkyl, substituted or unsubstituted aryl, substituted or unsubstituted heteroaryl, substituted or unsubstituted heterocycle, substituted or unsubstituted amino, and thiamine (vitamin B1), riboflavin (vitamin B2), niacin (vitamin B3), and pyridoxine (vitamin B6); or alternatively, Y¹ and Y² taken together are selected from the group consisting of sodium, potassium, lithium, magnesium, calcium, strontium, and barium;

Z¹ and Z² are independently NH or oxygen;

n is 0 or 1;

R¹ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H(R^{A})—(CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, (C₂-C₆)alkynyl, halogen, CN, NO₂, C(O)R^{C}, —C(O)OR^{C}, —C(O)NR^{C}₂, —C(═NR^{C})NR^{C}₂, —OR^{C}, —OC(O)(C₁-C₆)alkyl, —OC(O)(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

R^{A} is selected from the group consisting of —H, —(C₁-C₆)alkyl, —(CH₂)₃—NH—C(NH₂)(═NH), —CH₂C(═O)NH₂, —CH₂COOH, —CH₂SH, —(CH₂)₂C(═O)—NH₂, —(CH₂)₂COOH, —CH₂-(2-imidazolyl), —CH(CH₃)—CH₂—CH₃, —CH₂CH(CH₃)₂, —(CH₂)₄—NH₂, —(CH₂)₂—S—CH₃, phenyl, —CH₂-phenyl, —CH₂—OH, —CH(OH)—CH₃, —CH₂-(3-indolyl), —CH₂-(4-hydroxyphenyl), —CH(CH₃)₂, and —CH₂—CH₃;

R^{B} is hydrogen or —(C₁-C₈)alkyl;

each R^{C} is independently selected from the group consisting of hydrogen and —(C₁-C₈)alkyl;

R², R³, R⁴, and R⁵ are each independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

R⁶ and R⁷ are independently selected from the group consisting of hydrogen, —C(O)R′, —C(O)OR′, —C(O)NHR′, substituted or unsubstituted (C₁-C₈)alkyl, substituted or unsubstituted (C₁-C₈)cycloalkyl, substituted or unsubstituted aryl, substituted or unsubstituted heteroaryl, substituted or unsubstituted heterocycle, substituted or unsubstituted aryl(C₁-C₄)alkyl, and substituted or unsubstituted heterocycle(C₁-C₄)alkyl; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

R′ is selected from the group consisting of hydrogen, —(C₁-C₈)alkyl, —(C₁-C₈)cycloalkyl, aryl, heteroaryl, heterocycle, aryl(C₁-C₄)alkyl, and heterocycle(C₁-C₄)alkyl;

R″ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

Provided that when Z² is NH, the absolute configuration of C* is D or L, or a mixture of D and L.

Prototype product reduced nicotinamide/nicotinate ribonucleotide compounds include compounds having formula (II), or a salt thereof:

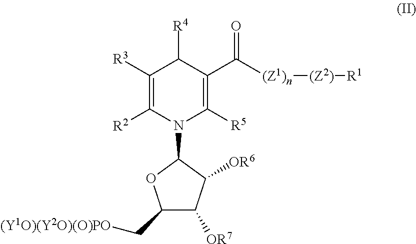

wherein Y¹, Y², Z¹, Z², n, R¹, R², R³, R⁴, R⁵, R⁶, and R⁷ are as defined above for the compounds having formula (I).

Appropriate starting materials include the unprotected riboside compounds having formula (1), or salts thereof:

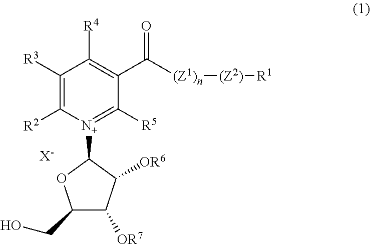

optionally wherein X⁻ as counterion is absent, or when X⁻ is present is selected from the group consisting of fluoride, chloride, bromide, iodide, formate, acetate, ascorbate, benzoate, carbonate, citrate, carbamate, formate, gluconate, lactate, methyl bromide, methyl sulfate, nitrate, phosphate, diphosphate, succinate, sulfate, and trifluoroacetate;

optionally wherein when X⁻ is absent, optionally the counterion is an internal salt;

Z¹ and Z² are independently NH or oxygen;

n is 0 or 1;

R¹ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

R^{A} is selected from the group consisting of —H, —(C₁-C₆)alkyl, —(CH₂)₃—NH—C(NH₂)(═NH), —CH₂C(—O)NH₂, —CH₂COOH, —CH₂SH, —(CH₂)₂C(═O)—NH₂, —(CH₂)₂COOH, —CH₂-(2-imidazolyl), —CH(CH₃)—CH₂—CH₃, —CH₂CH(CH₃)₂, —(CH₂)₄—NH₂, —(CH₂)₂—S—CH₃, phenyl, —CH₂-phenyl, —CH₂—OH, —CH(OH)—CH₃, —CH₂-(3-indolyl), —CH₂-(4-hydroxyphenyl), —CH(CH₃)₂, and —CH₂—CH₃;

R^{B} is hydrogen or —(C₁-C₈)alkyl;

each R^{C} is independently selected from the group consisting of hydrogen and —(C₁-C₈)alkyl;

R², R³, R⁴, and R⁵ are each independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

R⁶ and R⁷ are independently selected from the group consisting of hydrogen, —C(O)R′, —C(O)OR′, —C(O)NHR', substituted or unsubstituted (C₁-C₈)alkyl, substituted or unsubstituted (C₁-C₈)cycloalkyl, substituted or unsubstituted aryl, substituted or unsubstituted heteroaryl, substituted or unsubstituted heterocycle, substituted or unsubstituted aryl(C₁-C₄)alkyl, and substituted or unsubstituted heterocycle(C₁-C₄)alkyl; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

R′ is selected from the group consisting of hydrogen, —(C₁-C₈)alkyl, —(C₁-C₈)cycloalkyl, aryl, heteroaryl, heterocycle, aryl(C₁-C₄)alkyl, and heterocycle(C₁-C₄)alkyl;

R″ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and (C₁-C₆)alkylene-OR^{C};

provided that when Z² is NH, the absolute configuration of C* is D or L, or a mixture of D and L.

Appropriate starting materials further include the reduced nicotinamide/nicotinate unprotected riboside compounds having formula (2), or salts thereof:

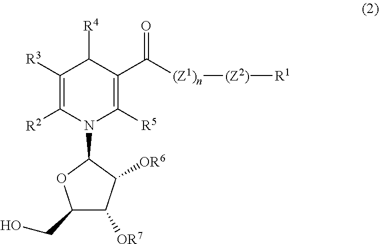

wherein Z¹, Z², n, R¹, R², R³, R⁴, R⁵, R⁶, and R⁷ are as defined above for the compounds having formula (1).

Generally, solvent-free conditions are employed, using appropriate mechano-chemical techniques as described.

A method of making a compound having formula (I) can include the steps of:

(a) providing a nicotinate/nicotinamide riboside compound or derivative having formula (1); (b) treating the compound or derivative having formula (1) with phosphorus oxychloride; (c) mechanically processing the components; (d) adding water to the mixture; (e) adjusting the pH with an aqueous base; (f) precipitating the compound having formula (I); and, optionally, (g) purifying and/or isolating the compound having formula (I). Mechanically processing includes one or more methods of agitation selected from the group consisting of grinding, mixing, milling, triturating, and liquid-assisted milling. The process described herein effects a chemoselective 5′-phosphorylation of an active hydroxyl group, such as an active hydroxyl group on the riboside moiety, in the absence of phosphate solvents.

In similar fashion, a method of making a compound having formula (II) can include the steps of:

(a) providing a reduced nicotinate/nicotinamide riboside compound or derivative having formula (2); (b) treating the compound or derivative having formula (2) with a base in the presence of a sub-molar (<1) equivalent amount of a polar organic solvent co-reagent; (c) mechanically processing the components in the presence of phosphorus oxychloride; (d) adding a neutralizing aqueous solution to the mixture; (e) filtering the mixture and adjusting the pH of the filtrate with an aqueous base if required; (f) precipitating the compound having formula (II); and, optionally, (g) purifying and/or isolating the compound having formula (II). The base can be selected from the group consisting of organic soluble bases, solid-supported bases, immobilized amine sorbents, and/or polymer and resin supported amine sorbents. Exemplary bases include morpholine, Hünig's Base (DIPEA), proton sponge, N,N,N′,N′-tetramethyl-1,8-naphthalenediamine, N,N,N′,N′-tetramethylethylenediamine , 1,8-diazobicyclo-[5.4.0]undec-7-ene, and Tröger's base.

Mechanically processing may include one or more methods of agitation selected from the group consisting of grinding, mixing, milling, triturating, and liquid-assisted milling. The process described herein effects a chemoselective 5′-phosphorylation of an active hydroxyl group, such as an active hydroxyl group on the riboside moiety, in the absence of phosphate solvents.

The polar organic solvent co-reagent employed in the above method of making a compound having formula (I) can be a polar organic solvent from among, for example, preferably, the Class 2 Residual Solvents listed in Table 2, or optionally, for non-human use, the Class 3 Residual Solvents listed in Table 3 in THE NATIONAL FORMULARY, UNITED STATES PHARMACOPEIA 30 <467> (U.S. PHARMACOPEIAL CONVENTION 2006) (USP 30 at <467>), incorporated by reference herein in its entirety.

An alternative method of making a compound having formula (I) can include the steps of:

(a) providing a nicotinate/nicotinamide riboside compound or derivative having formula (1); (b) treating the compound or derivative having formula (1) with phosphorus oxychloride in the presence of a sub-molar (<1) equivalent amount of a polar organic solvent co-reagent; (c) mechanically processing the components; (d) triturating the mixture, thus extracting excess phosphorus oxychloride and organic solvent co-reagent by adding with a small amount of diethyl ether; (e) adding iced water to the remaining solid mixture; (f) adjusting the pH with an aqueous base; (g) precipitating the compound having formula (I); and, optionally, (h) purifying and/or isolating the compound having formula (I). The stoichiometric equivalent amount of polar organic solvent co-reagent can be from about 0.5-molar to about 1.0-molar (in terms of phosphorylating agent). Mechanically processing may include one or more methods of agitation selected from the group consisting of grinding, mixing, milling, triturating, and liquid-assisted milling. The process described herein effects a chemoselective 5′-phosphorylation of an active hydroxyl group, such as an active hydroxyl group on the riboside moiety, in the absence of phosphate solvents.

The polar organic solvent co-reagent employed in the above method of making a compound having formula (I) can be a polar organic solvent from among, for example, preferably, the Class 2 Residual Solvents listed in Table 2, or optionally, for non-human use, the Class 3 Residual Solvents listed in Table 3 in THE NATIONAL FORMULARY, UNITED STATES PHARMACOPEIA 30 <467>(U.S. PHARMACOPEIAL CONVENTION 2006) (USP 30 at <467>), incorporated by reference herein in its entirety.

In similar fashion, an alternative method of making a compound having formula (II) can include the steps of:

(a) providing a reduced nicotinate/nicotinamide riboside compound or derivative having formula (2); (b) treating the compound or derivative having formula (2) with a base in the presence of a sub-molar equivalent amount of a polar organic solvent co-reagent; (c) mechanically processing the components in the presence of phosphorus oxychloride; (d) triturating the mixture thus extracting excess phosphorus oxychloride and organic solvent co-reagent by adding with a small amount of diethyl ether; (e) adding a neutralizing iced aqueous solution to the mixture; (f) filtering the mixture and adjusting the pH of the filtrate with an aqueous base if required; (g) precipitating the compound having formula (II); and, optionally, (h) purifying and/or isolating the compound having formula (II). The base can be selected from the group consisting of organic soluble bases, solid-supported bases, immobilized amine sorbents, and/or polymer and resin supported amine sorbents. Exemplary bases include morpholine, Hünig's Base (DIPEA), proton sponge, N,N,N′,N′-tetramethyl-1,8-naphthalenediamine, N,N,N′,N′-tetramethylethylenediamine, 1,8-diazobicyclo-[5.4.0]undec-7-ene, and Tröger's base. The stoichiometric equivalent amount of polar organic solvent co-reagent can be from about 0.5-molar to about 1.0-molar (in terms of phosphorylating agent). Mechanically processing may include one or more methods of agitation selected from the group consisting of grinding, mixing, milling, trituration, and liquid-assisted milling. The process described herein effects a chemoselective 5′-phosphorylation of an active hydroxyl group, such as an active hydroxyl group on the riboside moiety, in the absence of phosphate solvents.

The polar organic solvent co-reagent employed in the above methods of making a compound having formula (II) can be a polar organic solvent from among, for example, preferably, the Class 2 Residual Solvents listed in Table 2, or optionally, for non-human use, the Class 3 Residual Solvents listed in Table 3 in The National Formulary, UNITED STATES PHARMACOPEIA 30 <467>(U.S.. Pharmacopeial Convention 2006) (USP 30 at <467>), incorporated by reference herein in its entirety.

### BRIEF DESCRIPTION OF THE DRAWINGS

FIG. 1 depicts an HPLC chromatogram of Example 1 (nicotinamide mononucleotide, “NMN”), prepared in accordance with one embodiment of the described phosphorylation method.

FIG. 2 depicts a ¹H NMR spectrum of pure nicotinamide mononucleotide (“NMN”).

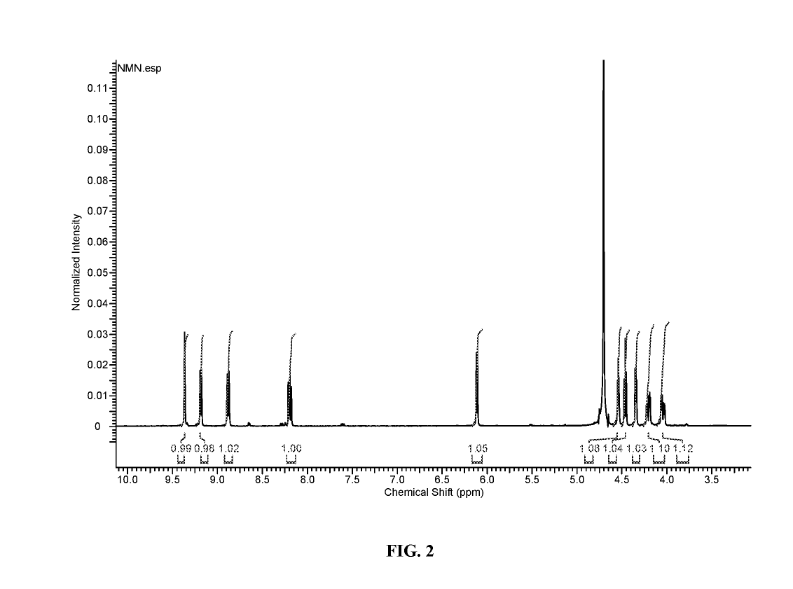

FIG. 3 depicts a ³¹P NMR spectrum of pure nicotinamide mononucletodie (“NMN”).

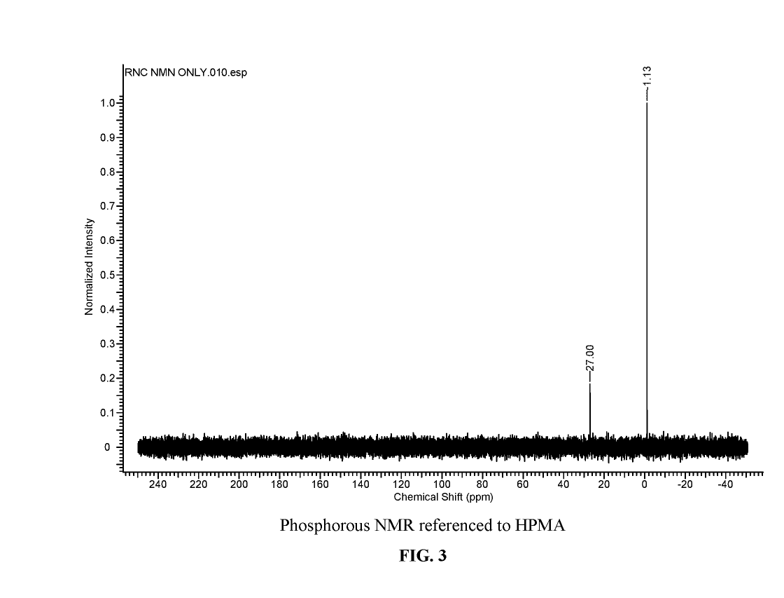

FIG. 4 depicts a ¹H NMR spectrum of the reaction product mixture for the procedure described in Example 1, “Method 2 (Scale-up),” performed in accordance with one embodiment of the described phosphorylation method.

FIG. 5 depicts a ¹³P NMR spectrum of the reaction product mixture for the procedure described in Example 1, “Method 2 (Scale-up),” performed in accordance with one embodiment of the described phosphorylation method.

FIG. 6 depicts a ¹³P NMR spectrum of the in situ reaction results of Example 6, performed in accordance with one embodiment of the described phosphorylation method.

FIG. 7 depicts a ¹H NMR spectrum of the crude mixture indicating conversion of reaction starting materials for the procedure described in Example 6, performed in accordance with one embodiment of the described phosphorylation method.

FIG. 8 depicts a ¹³C NMR spectrum of the reaction product for the procedure described in Example 6, performed in accordance with one embodiment of the described phosphorylation method.

FIG. 9 depicts a ¹H NMR spectrum demonstrating NR recovered after reaction for the procedure described in example 6, performed in accordance with one embodiment of the described phosphorylation method.

### DETAILED DESCRIPTION

In one aspect, the present invention surprisingly demonstrates the synthetic preparation of certain phosphorylated derivatives under solvent-free conditions for the first time. In a particular embodiment, the preparation of phosphorylated analogs of nicotinamide riboside (“NR”) and/or reduced or modified derivatives thereof, with an active hydroxyl group, are described. In another embodiment, the preparation of phosphorylated analogs of nicotinic acid riboside (“NAR”) and/or reduced or modified derivatives thereof, with an active hydroxyl group, are described. Solvent-free conditions are employed in combination with appropriate mechano-chemical techniques. This combination yields a process that is atom-efficient in terms of reagent equivalency, which bypasses the need for large amounts of polar solvents, and which is versatile in terms of limitations associated with reagents' solubility and reagents' mixing.

The mechanical processes described herein include grinding, mixing, milling, trituration, and/or liquid-assisted milling, and all related batch and continuous processes and enable efficient phosphorylation of many different compounds to produce derivatives, such as nucleotides and phosphorylated vitamins, under a phosphate solvent-free production protocol. The technique is applicable for the preparation of phosphorylated analogs of nicotinamide riboside (“NR”), nicotinic acid riboside (“NAR”), the reduced forms of the same (“NRH” and “NARH,” respectively), vitamins, etc.

The process for preparation of the phosphorylated derivatives involves, for example, grinding the respective components together in a mechano-chemical fashion utilizing mills such as planetary mills, etc.

The production of such derivatives using mechano-chemical principles has not been demonstrated before, particularly for producing biologically relevant nucleotides such as NMN, NAMN, and the like. The production technology has the ability to produce several other phosphorylated derivatives efficiently including isotopically labeled derivatives. As additional examples, thiamin (or thiamine) monophosphate, riboflavin monophosphate (FMN), phosphorylated vitamins, nucleoside monophosphate (“NMP”) such as adenosine monophosphate, and like species can be prepared efficiently using the synthetic process as described herein.

Additionally, the present production pathway addresses limitations of existing technologies to produce these compounds.

In an embodiment, the invention is directed to compounds having formula (I) or (II), and salts, hydrates, solvates, or prodrugs thereof, and processes for the preparation of said compounds.

The ribonucleotide compounds include compounds of formula (I), or a salt thereof:

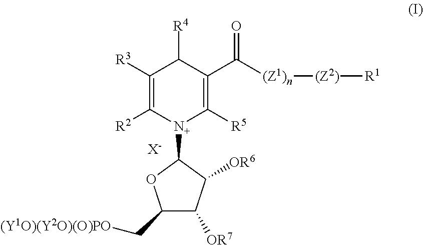

optionally wherein X⁻ as counterion is absent, or when X⁻ is present is selected from the group consisting of fluoride, chloride, bromide, iodide, formate, acetate, ascorbate, benzoate, carbonate, citrate, carbamate, formate, gluconate, lactate, methyl bromide, methyl sulfate, nitrate, phosphate, diphosphate, succinate, sulfate, trifluoromethanesulfonate, and trifluoroacetate; and,

optionally wherein when X⁻ is absent optionally the counterion is an internal salt;

optionally X⁻ is an anion of a substituted or unsubstituted carboxylic acid selected from a monocarboxylic acid, a dicarboxylic acid, or a polycarboxylic acid; and,

optionally X⁻ is an anion of a substituted monocarboxylic acid, further optionally an anion of a substituted propanoic acid (propanoate or propionate), or an anion of a substituted acetic acid (acetate), or an anion of a hydroxyl-propanoic acid, or an anion of 2-hydroxypropanoic acid (being lactic acid, the anion of lactic acid being lactate), or a trihaloacetate selected from trichloroacetate, tribromoacetate, and trifluoroacetate; and,

optionally X⁻ is an anion of an unsubstituted monocarboxylic acid selected from formic acid, acetic acid, propionic acid, or butyric acid, being formate, acetate, propionate, and butyrate, respectively; and,

optionally X⁻ is an anion of a substituted or unsubstituted amino acid, i.e., amino-monocarboxylic acid or an amino-dicarboxylic acid, optionally selected from glutamic acid and aspartic acid, being glutamate and aspartate, respectively; and,

optionally X⁻ is an anion of ascorbic acid, being ascorbate; and,

optionally X⁻ is a halide selected from fluoride, chloride, bromide, or iodide; and,

optionally X⁻ is an anion of a substituted or unsubstituted sulfonate, further optionally a trihalomethanesulfonate selected from trifluoromethanesulfonate, tribromomethanesulfonate, or trichloromethanesulfonate; and,

optionally X⁻ is an anion of a substituted or unsubstituted carbonate, further optionally hydrogen carbonate;

Y¹ and Y² are independently selected from the group consisting of hydrogen, sodium, potassium, lithium, substituted or unsubstituted (C₁-C₈)alkyl, substituted or unsubstituted (C₁-C₈)cycloalkyl, substituted or unsubstituted aryl, substituted or unsubstituted heteroaryl, substituted or unsubstituted heterocycle, substituted or unsubstituted amino, and thiamine (vitamin B1), riboflavin (B2), niacin (vitamin B3), and pyridoxine (vitamin B6); or alternatively, Y¹ and Y² taken together are selected from the group consisting of sodium, potassium, lithium, magnesium, calcium, strontium, and barium;

Z¹ and Z² are independently NH or oxygen;

n is 0 or 1;

R¹ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

R^{A} is selected from the group consisting of —H, —(C₁-C₆)alkyl, —(CH₂)₃—NH—C(NH₂)(═NH), —CH₂C(═O)NH₂, —CH₂COOH, —CH₂SH, —(CH₂)₂C(═O)—NH₂, —(CH₂)₂COOH, —CH₂-(2-imidazolyl), —CH(CH₃)—CH₂—CH₃, —CH₂CH(CH₃)₂, —(CH₂)₄—NH₂, —(CH₂)₂—S—CH₃, phenyl, —CH₂-phenyl, —CH₂—OH, —CH(OH)—CH₃, —CH₂-(3-indolyl), —CH₂—(4-hydroxyphenyl), —CH(CH₃)₂, and —CH₂—CH₃;

R^{B} is hydrogen or —(C₁-C₈)alkyl;

each R^{C} is independently selected from the group consisting of hydrogen and —(C₁-C₈)alkyl;

R², R³, R⁴, and R⁵ are each independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

R⁶ and R⁷ are independently selected from the group consisting of hydrogen, —C(O)R′, —C(O)OR′, —C(O)NHR′, substituted or unsubstituted (C₁-C₈)alkyl, substituted or unsubstituted (C₁-C₈)cycloalkyl, substituted or unsubstituted aryl, substituted or unsubstituted heteroaryl, substituted or unsubstituted heterocycle, substituted or unsubstituted aryl(C₁-C₄)alkyl, and substituted or unsubstituted heterocycle(C₁-C₄)alkyl; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

R′ is selected from the group consisting of hydrogen, —(C₁-C₈)alkyl, —(C₁-C₈)cycloalkyl, aryl, heteroaryl, heterocycle, aryl(C₁-C₄)alkyl, and heterocycle(C₁-C₄)alkyl;

R″ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

provided that when Z² is NH, the absolute configuration of C* is D or L, or a mixture of D and L.

Furthermore, anion X⁻ can be identical with one of the —O⁻ groups attached to one of Y¹ or Y² as an internal salt compound.

The reduced nicotinamide/nicotinate ribonucleotide compounds include compounds having formula (II), or a salt thereof:

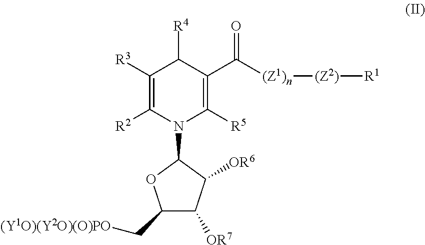

wherein Y¹, Y², Z¹, Z², n, R¹, R², R³, R⁴, R⁵, R⁶, and R⁷ are as defined above for the compounds having formula (II).

Appropriate starting materials include the unprotected riboside compounds of formula (1), or salts thereof:

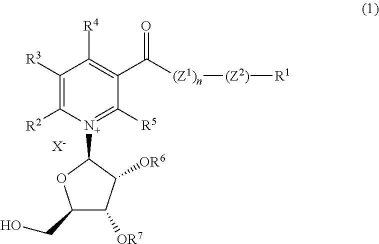

optionally wherein X⁻ as counterion is absent, or when X⁻ is present is selected from the group consisting of fluoride, chloride, bromide, iodide, formate, acetate, ascorbate, benzoate, carbonate, citrate, carbamate, formate, gluconate, lactate, methyl bromide, methyl sulfate, nitrate, phosphate, diphosphate, succinate, sulfate, and trifluoroacetate; and,

optionally wherein when X⁻ is absent optionally the counterion is an internal salt; and,

optionally X⁻ is an anion of a substituted or unsubstituted carboxylic acid selected from a monocarboxylic acid, a dicarboxylic acid, or a polycarboxylic acid; and,

optionally X⁻ is an anion of a substituted monocarboxylic acid, further optionally an anion of a substituted propanoic acid (propanoate or propionate), or an anion of a substituted acetic acid (acetate), or an anion of a hydroxyl-propanoic acid, or an anion of 2-hydroxypropanoic acid (being lactic acid, the anion of lactic acid being lactate), or a trihaloacetate selected from trichloroacetate, tribromoacetate, and trifluoroacetate; and,

optionally X⁻ is an anion of an unsubstituted monocarboxylic acid selected from formic acid, acetic acid, propionic acid, or butyric acid, being formate, acetate, propionate, and butyrate, respectively; and,

optionally X⁻ is an anion of a substituted or unsubstituted amino acid, i.e., amino-monocarboxylic acid or an amino-dicarboxylic acid, optionally selected from glutamic acid and aspartic acid, being glutamate and aspartate, respectively; and,

optionally X⁻ is an anion of ascorbic acid, being ascorbate; and,

optionally X⁻ is a halide selected from fluoride, chloride, bromide, or iodide; and,

optionally X⁻ is an anion of a substituted or unsubstituted sulfonate, further optionally a trihalomethanesulfonate selected from trifluoromethanesulfonate, tribromomethanesulfonate, or trichloromethanesulfonate; and,

optionally X⁻ is an anion of a substituted or unsubstituted carbonate, further optionally hydrogen carbonate;

Z¹ and Z² are independently NH or oxygen;

n is 0 or 1;

R¹ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of (C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

R^{A} is selected from the group consisting of —H, —(C₁-C₆)alkyl, —(CH₂)₃—NH—C(NH₂)(═NH), —CH₂C(═O)NH₂, —CH₂COOH, —CH₂SH, —(CH₂)₂C(—O)—NH₂, —(CH₂)₂COOH, —CH₂-(2-imidazolyl), —CH(CH₃)—CH₂—CH₃, —CH₂CH(CH₃)₂, —(CH₂)₄—NH₂, —(CH₂)₂—S—CH₃, phenyl, —CH₂-phenyl, —CH₂—OH, —CH(OH)—CH₃, —CH₂-(3-indolyl), —CH₂-(4-hydroxyphenyl), —CH(CH₃)₂, and —CH₂—CH₃;

R^{B} is hydrogen or —(C₁-C₈)alkyl;

each R^{C} is independently selected from the group consisting of hydrogen and —(C₁-C₈)alkyl;

R², R³, R⁴, and R⁵ are each independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

R⁶ and R⁷ are independently selected from the group consisting of hydrogen, —C(O)R′, —C(O)OR′, —C(O)NHR', substituted or unsubstituted (C₁-C₈)alkyl, substituted or unsubstituted (C₁-C₈)cycloalkyl, substituted or unsubstituted aryl, substituted or unsubstituted heteroaryl, substituted or unsubstituted heterocycle, substituted or unsubstituted aryl(C₁-C₄)alkyl, and substituted or unsubstituted heterocycle(C₁-C₄)alkyl ; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

R′ is selected from the group consisting of hydrogen, —(C₁-C₈)alkyl, —(C₁-C₈)cycloalkyl, aryl, heteroaryl, heterocycle, aryl(C₁-C₄)alkyl, and heterocycle(C₁-C₄)alkyl;

R″ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

provided that when Z² is NH, the absolute configuration of C* is D or L, or a mixture of D and L.

Appropriate starting materials further include the reduced nicotinamide/nicotinate unprotected riboside compounds having formula (2), or salts thereof:

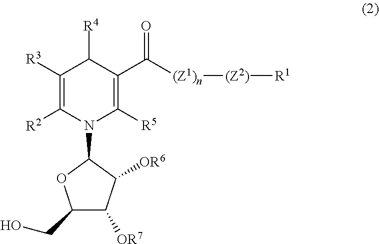

wherein Z¹, Z², n, R¹, R², R³, R⁴, R⁵, R⁶, and R⁷ are as defined above for the compounds having formula (2).

Definitions

As used in the specification and the appended claims, the singular forms of “a,” “an,” and “the” include plural referents unless the context clearly dictates otherwise.

As used herein, the terms “mechano-chemical mixing,” “mechanochemistry,” and “mechanical processing” refer to standard techniques known to those of ordinary skill in the art, in which chemical starting materials and/or reagents with disparate solubility properties are reacted, for example, by direct milling, liquid assisted-milling, triturating, mixing, or grinding, generally in the absence of solvents. Interchangeable terms may include “mechanico-chemical,” or the like. See F. Ravalico et al., Rapid synthesis of nucleotide pyrophosphate linkages in a ball mill, 9 ORG. BIOMOL. CHEM. 6496 (2011); Dritan Hasa et al., Cocrystal Formation through Mechanochemistry: From Neat and Liquid-Assisted Grinding to Polymer-Assisted Grinding, 127 ANGEWANDTE CHEMIE 7371 (2015); and references cited therein, all of which are incorporated by reference in their entireties.

The term “alkyl,” by itself or as part of another substituent means, unless otherwise stated, a straight, branched, or cyclic chain hydrocarbon (“cycloalkyl”) having the number of carbon atoms designated (i.e., C₁-C₆ means one to six carbons). Examples include methyl, ethyl, propyl, isopropyl, butyl, isobutyl, tert-butyl, pentyl, neopentyl, hexyl, cyclohexyl, and cyclopropyl. Most preferred are —(C₁-C₃)alkyl, particularly ethyl, methyl, and isopropyl.

The term “alkenyl,” employed alone or in combination with other terms, means, unless otherwise stated, a stable mono-unsaturated or di-unsaturated straight chain, the unsaturation meaning a carbon-carbon double bond (—CH═CH—), branched chain or cyclic hydrocarbon group having the stated number of carbon atoms. Examples include vinyl, propenyl, (allyl), crotyl, isopentenyl, butadienyl, 1,3-pentadienyl, 1,4-pentadienyl, cyclopentenyl, cyclopentadienyl, and the higher homologs and isomers. Functional groups representing an alkene are exemplified by —CH═CH—CH₂— and CH₂═CH—CH₂.

“Substituted alkyl” or “substituted alkenyl” means alkyl or alkenyl, respectively, as defined above, substituted by one, two, or three substituents. The substituents may, for example, be selected from the group consisting of halogen, —OH, —NH₂, —N(CH₃)₂, —C(═O)OH, —C(═O)O(C₁-C₄)alkyl, methoxy, ethoxy, trifluoromethyl, —C(═O)NH₂, —SO₂NH₂, —C(═NH)NH₂, —C═N, and —NO₂, preferably selected from halogen and —OH. Examples of substituted alkyls include, but are not limited to, 2,2-difluoromethyl, 2-carboxycyclopentyl, and 3-chloropropyl.

The term “alkynyl” employed alone or in combination with other terms, means, unless otherwise stated, a stable carbon-carbon triple bond-containing radical (—C≡C—), branched chain, or cyclic hydrocarbon group having the stated number of carbon atoms. Examples include ethynyl and propargyl.

The term “alkoxy” employed alone or in combination with other terms, means, unless otherwise stated, an alkyl group having the designated number of carbon atoms, as defined above, connected to the rest of the molecule via an oxygen atom, such as, for example, methoxy, ethoxy, 1-propoxy, 2-propoxy (“isopropoxy”), and the higher homologs and isomers. Preferred are —(C₁-C₃)alkoxy, particularly ethoxy and methoxy.

The terms “carbamyl” or “carbamoyl” mean the group —C(═O)NRR′, wherein R and R′ are independently selected from hydrogen or a hydrocarbyl functional group, or wherein R and R′ combined form a heterocycle. Examples of carbamyl groups include: —C(═O)NH₂ and —C(═O)N(CH₃)₂.

The term “cyano” refers to a —C≡N group.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur heteratoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂—CH₂—S(═O)—CH₃. Up to two heteroatoms may be consecutive, such as, for example, —CH₂NH—OCH₃ or —CH₂—CH₂—S—S—CH₃.

The terms “halo” or “halogen” by themselves or as part of another substituent mean, unless otherwise stated, a monovalent fluorine, chlorine, bromine, or iodine atom.

The term “nitro” refers to a —NO₂ group.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “aromatic” generally refers to a carbocycle or heterocycle having one or more polyunsaturated rings having aromatic character (i.e., having (4n+2) delocalized π (pi) electrons where n is an integer).

The term “aryl,” employed alone or in combination with other terms, means, unless otherwise stated, a carbocyclic aromatic system containing one or more rings (typically one, two, or three rings) wherein such rings may be attached together in a pendant manner, such as a biphenyl, or may be fused, such as naphthalene. Examples include phenyl; anthracyl; and naphthyl. Preferred are phenyl and naphthyl, most preferred is phenyl.

The term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom independently selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure.

The term “heteroaryl” or “heteroaromatic” refers to a heterocyclic having aromatic character. Similarly, the term “heteroaryl(C₁-C₃)alkyl” means a functional group wherein a one to three carbon alkylene chain is attached to a heteroaryl group, e.g., —CH₂—CH₂pyridyl. The term “substituted heteroaryl(C₁-C₃)alkyl” means a heteroaryl(C₁-C₃)alkyl functional group in which the heteroaryl group is substituted. A polycyclic heteroaryl may include fused rings. Examples include indole, 1H-indazole, 1H-pyrrolo[2,3-b]pyridine, and the like. A polycyclic heteroaryl may include one or more rings that are partially saturated. Examples include indoline, tetrahydroquinoline, and 2,3-dihydrob enzofuryl .

Examples of non-aromatic heterocycles include monocyclic groups such as: aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, piperazine, N-methylpiperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

Examples of heteroaryl groups include: pyridyl; pyrazinyl; pyrimidinyl, particularly 2- and 4-pyrimidinyl; pyridazinyl; thienyl; furyl; pyrrolyl, particularly 2-pyrrolyl; imidazolyl; thiazolyl; oxazolyl; pyrazolyl, particularly 3- and 5-pyrazolyl; isothiazolyl; 1,2,3-triazolyl; 1,2,4-triazolyl; 1,3,4-triazolyl; tetrazolyl; 1,2,3-thiadiazolyl; 1,2,3-oxadiazolyl; 1,3,4-thiadiazolyl; and 1,3,4-oxadiazolyl .

Polycyclic heterocycles include both aromatic and non-aromatic polycyclic heterocycles. Examples of polycyclic heterocycles include: indolyl, particularly 3-, 4-, 5-, 6-, and 7-indolyl; indolinyl; indazolyl, particularly 1H-indazol-5-yl; quinolyl; tetrahydroquinolyl; isoquinolyl, particularly 1- and 5-isoquinolyl; 1,2,3,4-tetrahydroisoquinolyl; cinnolyl; quinoxalinyl, particularly 2- and 5-quinoxalinyl; quinazolinyl; phthalazinyl; naphthyridinyl, particularly 1,5- and 1,8-naphthyridinyl; 1,4-benzodioxanyl; coumaryl; dihydrocoumaryl; benzofuryl, particularly 3-, 4-, 5-, 6-, and 7-b enzofuryl; 2,3 -dihydrobenzofuryl; 1,2-benzisoxazolyl; benzothienyl, particularly 3-, 4-, 5-, 6-, and 7-benzothienyl; benzoxazolyl; benzothiazolyl, particularly 2- and 5-benzothiazolyl; purinyl; benzimidazolyl, particularly 2-benzimidazolyl; benztriazolyl; thioxanthinyl; carbazolyl; carbolinyl; acridinyl; pyrrolizidinyl; pyrrolo[2,3-b]pyridinyl, particularly 1H-pyrrolo[2,3-b]pyridine-5-yl; and quinolizidinyl. Particularly preferred are 4-indolyl, 5-indolyl, 6-indolyl, 1H-indazol-5-yl, and 1H-pyrrolo[2,3-b]pyridine-5-yl.

The aforementioned listing of heterocyclic and heteroaryl moieties is intended to be representative and not limiting.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position.

D-Ribose stereochemistry has been indicated in formulas (I) and (II). It is understood that the configuration at the anomeric carbon can be reversed (i.e. L-), or can be a mixture of D- and L-.

Synthetic preparation of Phosphorylated compounds having formulas (I) and (II).

In one embodiment, a method of making a compound having formula (I) is provided. The compound having formula (I) may be prepared by a process comprising:

(a) providing a nicotinate/nicotinamide riboside compound or derivative having formula (1);

(b) treating the compound or derivative having formula (1) with phosphorus oxychloride or another suitable phosphorylating agent;

(c) mechanically processing the components;

(d) adding water to the mixture;

(e) adjusting the pH with an aqueous base;

(f) precipitating the compound having formula (I); and

optionally, (g) purifying and/or isolating the compound having formula (I).

Mechanically processing may include one or more methods of agitation selected from the group consisting of grinding, mixing, milling, triturating, and liquid-assisting milling. Mixing and/or milling may be performed between about 5 Hz and about 50 Hz for about 1 min to about 500 min, preferably between about 10 Hz and 40 Hz for about 15 min to about 180 min, and most preferably between about 20 Hz and 30 Hz for about 60 min to about 120 min. Grinding may be performed between about 50 RPM and about 200 RPM, preferably between about 75 RPM and about 150 RPM, and most preferably between about 100 RPM and about 130 RPM.

The process described herein effects a chemoselective 5′-phosphorylation of an active hydroxyl group, such as an active hydroxyl group on the riboside moiety, in the absence of phosphate solvents.

In another embodiment, a method of making a compound having formula (II) is provided including the steps of:

(a) providing a reduced form of nicotinate/nicotinamide riboside compound or derivative having formula (2);

(b) treating the compound or derivative having formula (2) with a base in the presence of a sub-molar (<1) equivalent amount of a polar organic solvent co-reagent;

(c) mechanically processing the components in the presence of phosphorus oxychloride or another suitable phosphorylating agent;

(d) adding a neutralizing aqueous solution to the mixture;

(e) filtering the mixture and adjusting the pH of the filtrate with an aqueous base if required;

(f) precipitating the compound having formula (II); and

optionally, (g) purifying and/or isolating the compound having formula (II).

The base can be selected from the group consisting of organic soluble bases, solid-supported bases, immobilized amine sorbents, and/or polymer and resin supported amine sorbents. Exemplary bases include morpholine, Hünig' s Base (DIPEA), proton sponge, N,N,N′,N′-tetramethyl-1,8-naphthalenediamine, N,N,N′,N ′-tetramethylethylenediamine, 1,8-diazobicyclo-[5.4.0]undec-7-ene, and Tröger's base.

Mechanically processing may include one or more methods of agitation selected from the group consisting of grinding, mixing, milling, triturating, and liquid-assisting milling. Mixing and/or milling may be performed between about 5 Hz and about 50 Hz for about 1 min to about 500 min, preferably between about 10 Hz and 40 Hz for about 15 min to about 180 min, and most preferably between about 20 Hz and 30 Hz for about 60 min to about 120 min. Grinding may be performed between about 50 RPM and about 200 RPM, preferably between about 75 RPM and about 150 RPM, and most preferably between about 100 RPM and about 130 RPM.

The process described herein also effects a chemoselective 5′-phosphorylation of an active hydroxyl group, such as an active hydroxyl group on the riboside moiety, in the absence of phosphate solvents.

The polar organic solvent co-reagent employed in the above method of making a compound having formula (I) can be a polar organic solvent from among, for example, preferably, the Class 2 Residual Solvents listed in Table 2, or optionally, for non-human use, the Class 3 Residual Solvents listed in Table 3 in USP 30 at <467>.

In another embodiment, an alternative method of making a compound having formula (I) can include the steps of:

(a) providing a nicotinate/nicotinamide riboside compound or derivative having formula (¹);

(b) treating the compound or derivative having formula (1) with phosphorus oxychloride in the presence of a sub-molar (<1) equivalent amount of a polar organic solvent co-reagent;

(c) mechanically processing the components;

(d) triturating the mixture, thus extracting excess phosphorus oxychloride and organic solvent co-reagent by adding with a small amount of diethyl ether;

(e) adding iced water to the remaining solid mixture;

(f) adjusting the pH with an aqueous base;

(g) precipitating the compound having formula (I); and

optionally, (h) purifying and/or isolating the compound having formula (I).

The stoichiometric equivalent amount of polar organic solvent co-reagent can be from about 0.5-molar to about 1.0-molar (in terms of phosphorylating agent). Mechanically processing may include one or more methods of agitation selected from the group consisting of grinding, mixing, milling, triturating, and liquid-assisted milling. Mixing and/or milling may be performed between about 5 Hz and about 50 Hz for about 1 min to about 500 min, preferably between about 10 Hz and 40 Hz for about 15 min to about 180 min, and most preferably between about 20 Hz and 30 Hz for about 60 min to about 120 min. Grinding may be performed between about 50 RPM and about 200 RPM, preferably between about 75 RPM and about 150 RPM, and most preferably between about 100 RPM and about 130 RPM.

The process described herein effects a chemoselective 5′-phosphorylation of an active hydroxyl group, such as an active hydroxyl group on the riboside moiety, in the absence of phosphate solvents.

The polar organic solvent co-reagent employed in the above method of making a compound having formula (I) can be a polar organic solvent from among, for example, preferably, the Class 2 Residual Solvents listed in Table 2, or optionally, for non-human use, the Class 3 Residual Solvents listed in Table 3 in USP 30 at <467>.

In another embodiment, an alternative method of making a compound having formula (II) can include the steps of:

(a) providing a reduced nicotinate/nicotinamide riboside compound or derivative having formula (2);

(b) treating the compound or derivative having formula (2) with a base in the presence of a sub-molar (<1) equivalent amount of a polar organic solvent co-reagent;

(c) mechanically processing the components in the presence of phosphorus oxychloride or another suitable phosphorylating agent;

(d) triturating the mixture thus extracting excess phosphorus oxychloride and organic solvent co-reagent by adding with a small amount of diethyl ether;

(e) adding a neutralizing iced aqueous solution to the mixture;

(f) filtering the mixture and adjusting the pH of the filtrate with an aqueous base if required;

(g) precipitating the compound having formula (II); and

optionally, (h) purifying and/or isolating the compound having formula (II).

The base can be selected from the group consisting of organic soluble bases, solid-supported bases, immobilized amine sorbents, and/or polymer and resin supported amine sorbents. Exemplary bases include morpholine, Hi.inig' s Base (DIPEA), proton sponge, N,N,N′,N′-tetramethyl-1,8-naphthalenediamine, N,N,N′,N′-tetramethylethylenediamine, 1,8-diazobicyclo-[5.4.0]undec-7-ene, and Tröger's base. The stoichiometric equivalent amount of polar organic solvent co-reagent can be from about 0.5-molar to about 1.0-molar (in terms of phosphorylating agent). Mechanically processing may include one or more methods of agitation selected from the group consisting of grinding, mixing, milling, triturating, and liquid-assisted milling. Mixing and/or milling may be performed between about 5 Hz and about 50 Hz for about 1 min to about 500 min, preferably between about 10 Hz and 40 Hz for about 15 min to about 180 min, and most preferably between about 20 Hz and 30 Hz for about 60 min to about 120 min. Grinding may be performed between about 50 RPM and about 200 RPM, preferably between about 75 RPM and about 150 RPM, and most preferably between about 100 RPM and about 130 RPM.

The process described herein effects a chemoselective 5′-phosphorylation of an active hydroxyl group, such as an active hydroxyl group on the riboside moiety, in the absence of phosphate solvents.

The polar organic solvent co-reagent employed in the above method of making a compound having formula (I) can be a polar organic solvent from among, for example, preferably, the Class 2 Residual Solvents listed in Table 2, or optionally, for non-human use, the Class 3 Residual Solvents listed in Table 3 in USP 30 at <467>.

It is understood that the pH can be adjusted to the isoelectric point of the product compound(s), or near neutral pH. Precipitation of the product compound(s) can be carried out using an appropriate water-miscible or other generally non-toxic solvent.

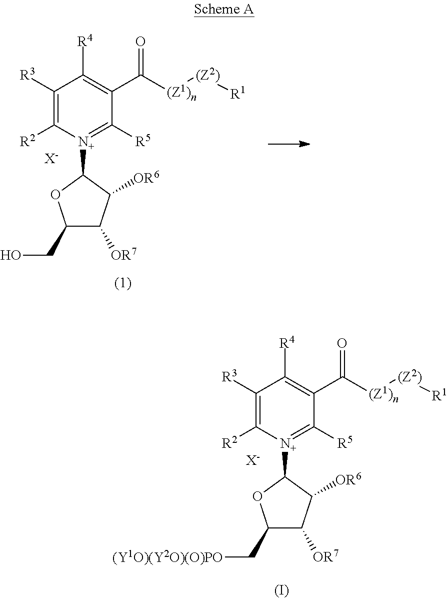

The chemoselective synthesis of the compound having formula (I) is shown above in Scheme A.

As discussed above, the existing prior art approaches, for the most part, utilize enzymatic and solvent-mediated approaches to prepare the phosphorylated derivatives. Such processes are cumbersome, inefficient, and not scalable. One reference from one of the present inventors describes synthesizing nucleotide phyrophosphate linkages in a ball mill. See Ravalico et al. (2011), as cited above, herein incorporated by reference.

Immobilized amine sorbents show similar reactions to liquid amines in the typical absorption process, with the added advantages that solids are easier to handle and that they do not give rise to the corrosion problems caused by the circulation of highly basic solutions. It is possible to coat solid polymers with liquid amines to combine the high surface area of the polymeric support with the CO₂ removal efficiency of a liquid amine. Polyethyleneimine (“PEI”) and diethanolamine (“DEA”) are two amines that can be applied to support surfaces. Satyapal and others developed a CO₂ sorbent in the form of 300-600 micron acrylic based polymer beads coated with a liquid amine, capable of removing a maximum capacity of ˜8 wt % CO₂ from an air stream and with no loss of performance over hundreds of adsorption-desorption cycles. Ethylenediamine (“EDA”) and tetraethylenepentamine (“TEPA”) have been demonstrated as efficient bases when immobilized within the pores of high surface area poly(methylmethylacrylate) (“PMMA”) solid beads. The two-ring sterically hindered amidine base 1,8-diazobicyclo-[5.4.0]undec-7-ene (“DBU”) has been demonstrated as an efficient base when immobilized on polystyrene and PMMA beads. DEA-supported amberlite acrylic ester resin has also been shown energetically effective compared with 30 wt % DEA in aqueous solution.

One suitable phosphorylating agent is phosphorus oxychloride (POCl₃). Other suitable phosphorylating agents (or phosphorus reagent systems) including compounds having formula P(O)Cl(OR^{X})(OR^{Y}) that include CAS Numbers 2524-64-3, 6609-64-9, 814-49-3, 14254-41-2, 2574-25-6, 813-77-4, 1499-17-8, 2510-89-6, 819-43-2, 5381-98-6, 538-37-4, 57188-46-2, 81639-99-8, 17672-53-6, 4090-55-5, 17776-78-2, 6630-13-3, 56119-60-9, 77075-54-8, 89104-48-3, 6546-97-0, 6630-15-5, 16383-57-6, 381-44-2, 124648-60-8, 17788-08-8, 58377-73-4, 6630-14-4, 17158-87-1, 17677-92-8, 51103-92-5, 52258-06-7, 56623-07-5, 58377-74-5, 85363-77-5, 112966-13-9, 167907-25-7, 179695-78-4, 877458-32-7, 1424937-89-2, 1424939-04-7, 2035-83-8, 127164-51-6, 6719-79-5, 59819-52-2, 69919-18-2, 77181-80-7, 4040-23-7, 6533-33-1, 6719-82-0, 6719-84-2, 22939-24-8, 27315-40-8, 28888-24-6, 61550-37-6, 73992-66-2, 86531-53-5, 96357-53-8, 108249-87-2, 343863-91-2, 875893-99-5, 714-87-4, 6087-94-1, 13674-83-4, 56883-17-1, 88805-00-9, 92401-83-7, 93115-98-1, 120628-26-4, 130312-59-3, 315179-27-2, 1388636-60-9, 1388636-61-0; and compounds having formula P(O)C1₂(OR^{z}) that include CAS Numbers 770-12-7, 1498-51-7, 15074-54-1, 777-52-6, 677-24-7, 772-79-2, 4167-02-6, 1455-05-6, 31651-76-0, 53676-22-5, 18868-46-7, 53676-18-9, 940-18-1, 84681-46-9, 878-17-1, 105053-57-4, 149864-64-2, 6964-36-9, 18350-98-6, 53676-17-8, 60223-35-0, 25359-51-7, 2035-84-9, 2196-02-3, 382608-79-9, 775-08-6, 30333-08-5, 1479-10-3, 2213-71-0, 5305-82-8, 5995-77-7, 13674-82-3, 13825-97-3, 17788-07-7, 19430-76-3, 19430-77-4, 20056-41-1, 20464-68-0, 31735-82-7, 36196-79-9, 41998-90-7, 52198-45-5, 53121-39-4, 53121-41-8, 99884-77-2, 105053-58-5, 125440-36-0, 140468-02-6, 140468-03-7, 184528-42-5, 870673-87-3, 916893-01-1, 1498-52-8, 20464-67-9, 38135-34-1, 41240-73-7, 62485-00-1, 78840-91-2, 313946-12-2, 1242826-74-9. R^{x}, R^{Y}, and R^{z} may be the same or different, and include, but are not limited to, simple alkyl.

The present invention further embraces isolated compounds according to formulas (I) and (II). The expression “isolated compound” refers to a preparation of a compound having formula (I) or (II), or a mixture of compounds according to formulas (I) and/or (II), wherein the isolated compound has been separated from the reagents used, and/or byproducts formed, in the synthesis of the compound or compounds. “Isolated” does not mean that the preparation is technically pure (homogeneous), but that it has sufficient purity.

The compounds of the invention, and intermediates, may be isolated from their reaction mixtures and purified by standard techniques such as filtration, liquid-liquid extraction, solid phase extraction, distillation, recrystallization, or chromatography, including flash column chromatography, preparative TLC, HPTLC, HPLC, or rp-HPLC. One preferred method for purification of the compounds according to formula (I) or (II) or salts thereof comprises crystallizing the compound or salt from a solvent to form, preferably, a crystalline form of the compounds or salts thereof. Following crystallization, the crystallization solvent is removed by a process other than evaporation, for example filtration or decanting, and the crystals are then preferably washed using pure solvent (or a mixture of pure solvents). Preferred solvents for crystallization include water; alcohols, particularly alcohols containing up to four carbon atoms, such as methanol, ethanol, isopropanol, and butan-l-ol, butan-2-ol, and 2-methyl-2-propanol; ethers, for example diethyl ether, diisopropyl ether, t-butyl methyl ether, 1,2-dimethoxyethane, tetrahydrofuran, and 1,4-dioxane; carboxylic acids, for example formic acid and acetic acid; hydrocarbon solvents, for example pentane, hexane, toluene; and mixtures thereof, particularly aqueous mixtures such as aqueous ethanol. Pure solvents, preferably at least analytical grade, and more preferably pharmaceutical grade are preferably used. In a preferred embodiment of the processes of the invention, the products are so isolated. In the compounds of the invention according to formula (I) or (II) or salts thereof, and pharmaceutical compositions thereof, the compounds according to formula (I) or (II) or salts thereof are preferably in or prepared from a crystalline form, preferably prepared according to such a process. Alternatively, the compounds according to formula (I) or (II) or salts thereof can be isolated using lyophilization or freeze-drying techniques, following ion-exchange purification, thus avoiding use of non-aqueous solvents.

The synthetic methods described above reflect a convergent synthesis strategy. Thus, two components may be synthesized and elaborated separately prior to condensing or coupling the compounds to form the target compounds. These convergent synthetic schemes allow for arrangement of the assembly steps of the backbone of the target compounds and derivatization of derivatizable functionalities to accommodate functional group sensitivity and/or to allow for functional groups or elements to be introduced either before or after the assembly of the backbone of the target compounds via the condensation or coupling reactions described.

It will be appreciated by one skilled in the art that certain aromatic substituents in compounds of the invention, intermediates used in the processes above, or precursors thereto, may be introduced by employing aromatic substitution reactions to introduce or replace a substituent, or by using functional group transformations to modify an existing substituent, or a combination thereof. Such reactions may be effected either prior to or immediately following the processes mentioned above, and are included as part of the process aspect of the invention. The reagents and reaction conditions for such procedures are known in the art. Specific examples of procedures that may be employed include, but are not limited to, electrophilic functionalization of an aromatic ring, for example via nitration, halogenations, or acylation; transformation of a nitro group to an amino group, for example via reduction, such as by catalytic hydrogenation; acylation, alkylation, or sulfonylation of an amino or hydroxyl group; replacement of an amino group by another functional group via conversion to an intermediate diazonium salt followed by nucleophilic or free radical substitution of the diazonium salt; or replacement of a halogen by another group, for example via nucleophilic or organometallically-catalyzed substitution reactions.

Additionally, in the aforesaid processes, certain functional groups that would be sensitive to the reaction conditions may be protected by protecting groups. A protecting group is a derivative of a chemical functional group that would otherwise be incompatible with the conditions required to perform a particular reaction that, after the reaction has been carried out, can be removed to regenerate the original functional group, which is thereby considered to have been “protected.” Any chemical functionality that is a structural component of any of the reagents used to synthesize compounds of this invention may be optionally protected with a chemical protecting group if such a protecting group is useful in the synthesis of compounds of this invention. The person skilled in the art knows when protecting groups are indicated, how to select such groups, and processes that can be used for selectively introducing and selecting removing them, because methods of selecting and using protecting groups have been extensively documented in the chemical literature. Techniques for selecting, incorporating, and removing chemical protecting groups may be found, for example, in THEODORA W. GREENE & PETER G. M. WUTS, PROTECTIVE GROUPS IN ORGANIC SYNTHESIS (John Wiley & Sons, Inc. 1999), the entire disclosure of which is incorporated herein by reference.

In addition to use of a protecting group, sensitive functional groups may be introduced as synthetic precursors to the functional groups desired in the intermediate or final product. An example of this is an aromatic nitro (—NO₂) group. The aromatic nitro group does not undergo any of the nucleophilic reactions of an aromatic amino group. However, the nitro group can serves as the equivalent of a protected amino group because it is readily reduced to the amino group under mild conditions that are selective for the nitro group over most other functional groups.

It will be appreciated by one skilled in the art that the processes described are not the exclusive means by which compounds of the invention may be synthesized and that an extremely broad repertoire of synthetic organic reactions is available to be potentially employed in synthesizing compounds of the invention. The person skilled in the art knows how to select and implement appropriate synthetic routes. Suitable synthetic methods may be identified by reference to the literature, including reference sources such as COMPREHENSIVE ORGANIC SYNTHESIS (B.M. Trost & I. Fleming eds., Pergamon Press 1991); COMPREHENSIVE ORGANIC FUNCTIONAL GROUP TRANSFORMATIONS (A.R. Katritzky, O. Meth-Cohn, & C.W. Rees eds., Pergamon Press 1996); COMPREHENSIVE ORGANIC FUNCTIONAL GROUP TRANSFORMATIONS II (A.R. Katritzky & R.J.K. Taylor eds., 2d ed., Elsevier 2004); COMPREHENSIVE HETEROCYCLIC CHEMISTRY (A.R. Katritzky & C.W. Rees eds., Pergamon Press 1984); COMPREHENSIVE HETEROCYCLIC CHEMISTRY II (A.R. Katritzky, C.W. Rees, & E.F.V. Scriven eds., Pergamon Press 1996); and J. MARCH, ADVANCED ORGANIC CHEMISTRY (4th ed., John Wiley & Sons, Inc. 1992).

Salts of Compounds According to the Invention

The compounds of the present invention may take the form of salts. The term “salts” embraces addition salts of free acids or free bases that are compounds of the invention. The term “pharmaceutically acceptable salt” refers to salts that possess toxicity profiles within a range that affords utility in pharmaceutical applications.

Suitable pharmaceutically acceptable acid solution salts may be prepared from an inorganic acid or from an organic acid. Examples of inorganic acids include hydrochloric, hydrobromic, hydroiodic, nitric, carbonic, sulfuric, and phosphoric acids. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoroacetic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, and galacturonic acid. In the present examples of compounds having formula (I) or (II), i.e., compounds containing amino groups, pyridine or reduced pyridine, said compounds can be isolated as salts of inorganic acids or strong organic acids, e.g., hydrochloric acid or trifluoroacetic acid.

Suitable pharmaceutically acceptable base addition salts of compounds of the invention include, for example, metallic salts including alkali metal, alkaline earth metal, and transition metal salts such as, for example, calcium, magnesium, potassium, sodium, and zinc salts. Further, base addition salts of compounds of the invention include, for example, ammonium salts. Pharmaceutically acceptable base addition salts also include organic salts made from basic amines such as, for example, N,N-dibenzylethylenediamine, chloroprocaine, choline, diethanolamine, ethylenediamine, meglumine (N-methylglucamine), tromethamine (tris(hydroxymethyl)aminomethane), and procaine.

All of these salts may be prepared by conventional means from the corresponding compounds having formula (I) or (II) by reacting, for example, the appropriate acid or base with the compounds having formula (I) or (II). Preferably, the salts are in crystalline form, or alternatively in dried or freeze-dried form. The person skilled in the art will know how to prepare and select suitable salt forms for example, as described in P.H. STAHL & C.G. WERMUTH, HANDBOOK OF PHARMACEUTICAL SALTS: PROPERTIES, SELECTION, AND USE (Wiley-VCH 2002).

The nutraceutical compositions of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluents, or excipient that is compatible with the other ingredients of the formulation and not deleterious to the user. In accordance with one embodiment, suitable nutraceutically acceptable carriers can include ethanol, aqueous ethanol mixtures, water, fruit, and/or vegetable juices, and combinations thereof.

Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films, or oral dissolvable strips can be used. Other useful delivery systems comprise oral or nasal sprays or inhalers, and the like.

For oral administration, a compound having formula (I) or (II) may be further combined with one or more solid inactive ingredients for the preparation of tablets, capsules, pills, powders, granules, or other suitable dosage forms. For example, the active agent may be combined with at least one excipient such as fillers, binders, humectants, disintegrating agents, solution retarders, absorption accelerators, wetting agents, absorbents, or lubricating agents. Other useful excipients include magnesium stearate, calcium stearate, mannitol, xylitol, sweeteners, starch, carboxymethylcellulose, microcrystalline cellulose, silica, gelatin, silicon dioxide, and the like.

The components of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parental use. Such pharmaceutical compositions and unit dosage forms thereof many comprise conventional ingredients in conventional proportions, with or without additional active compounds or principles, and such unit dosage forms may contain any suitable effective amount of the active ingredient commensurate with the intended daily dosage range to be employed.

The components of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active component. In tablets, the active component is mixed with the carrier having the necessary binding capacity in suitable proportions and compacted in the shape and size desired.

The powders and tablets preferably contain from five or ten to about seventy percent of the active compound(s). Suitable carriers are microcrystalline cellulose, sugar, lactose, pectin, dextrin, starch, gelatin, tragacanth, methylcellulose, sodium carboxymethylcellulose, a low melting wax, cocoa butter, and the like, and other excipients may include magnesium stearate, stearic acid, talc, silicon dioxide, etc. The term “preparation” is intended to include the formulation of the active compound with encapsulating material as carrier providing a capsule in which the active component, with or without carriers, is surrounded by a carrier, which is thus in association with it. Tablets, powders, capsules, pills, sachets, and lozenges are included. Tablets, powders, capsules, pills, sachets, and lozenges can be used as solid forms suitable for oral administration.

Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g., by injection, for example bolus injection or continuous infusion) and may be presented in unit dose for example in ampoules, pre-filled syringes, small volume infusion, or in multi-dose containers with an added preservative. The compositions may take such forms as suspensions, solutions, or emulsions in oily or aqueous vehicles, and may contain formulation agents such as suspending, stabilizing, and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

Compositions suitable for topical administration in the mouth includes lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

Solutions or suspensions are applied directly to the nasal cavity by conventional means, for example, with a dropper, pipette, or spray. The compositions may be provided in single or multi-dose form. In compositions intended for administration to the respiratory tract, including intranasal compositions, the compound will generally have a small particle size, for example, of the order of 5 microns or less. Such a particle size may be obtained by means known in the art, for example by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenges itself, or it can be the appropriate number of any of these in packaged form.

Tablets, capsules, and lozenges for oral administration and liquids for oral use are preferred compositions. Solutions or suspensions for application to the nasal cavity or to the respiratory tract are preferred compositions. Transdermal patches for topical administration to the epidermis are preferred.

Further details on techniques for formulation and administration may be found in the latest edition of Remington's Pharmaceutical Sciences (Mack Publishing Co., Easton, Pa.).

Solid nutritional compositions for oral administration may optionally contain, in addition to the above enumerated nutritional composition ingredients or compounds: carrier materials such as corn starch, gelatin, acacia, microcrystalline cellulose, kaolin, dicalcium phosphate, calcium carbonate, sodium chloride, alginic acid, and the like; disintegrators, including microcrystalline cellulose, aliginic acid, and the like; binders including acacia, methylcellulose, sodium carboxymethylcellulose, polyvinylpyrrolidone, hydroxypropyl methylcellulose, ethyl cellulose, and the like; and lubricants such as magnesium stearate, stearic acid, silicone fluid, talc, waxes, oils, colloidal silica, and the like. The usefulness of such excipients is well known in the art.

Liquid nutritional compositions for oral administration in connection with a method for preventing and/or treating inflammation, colds, and/or flue can be prepared in water or other aqueous vehicles. In addition to the above enumerated ingredients or compounds, liquid nutritional compositions can include suspending agents such as, for example, methylcellulose, alginates, tragacanth, pectin, kelgin, carrageenan, acacia, polyvinylpyrrolidone, polyvinyl alcohol, and the like. The liquid nutritional compositions can be in the form of a solution, emulsion, syrup, gel, or elixir including or containing, together with the above enumerated ingredients or compounds, wetting agents, sweeteners, and coloring and flavoring agents. Various liquid and powder nutritional compositions can be prepared by conventional methods. Various ready-to-drink formulations (“RTDs”) are contemplated.

Routes of Administration

The compositions may be administered by any suitable route, including but not limited to oral, sublingual, buccal, ocular, pulmonary, rectal, and parenteral administration, or as an oral or nasal spray (e.g., inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of a pharmaceutical composition in the body of the patient in a controlled formulation, with systemic or local release of the drug to occur at a later time. For example, the drug may be localized in a depot for controlled release to the circulation, or for release to a local site.

Pharmaceutical compositions of the invention may be those suitable for oral, rectal, bronchial, nasal, pulmonal, topical (including buccal and sub-lingual), transdermal, vaginal, or parenteral (including cutaneous, subcutaneous, intramuscular, intraperitoneal, intravenous, intraarterial, intracerebral, intraocular injection, or infusion) administration, or those in a form suitable for administration by inhalation or insufflations, including powders and liquid aerosol administration, or by sustained release systems. Suitable examples of sustained release systems include semipermeable matrices of solid hydrophobic polymers containing the compound of the invention, which matrices may be in the form of shaped artices, e.g., films or microcapsules.

The methods described above may be further understood in connection with the following Examples. In addition, the following non-limiting examples are provided to illustrate the invention. The illustrated synthetic pathways are applicable to other embodiments of the invention. The synthetic procedures described as general methods describe what it is believed will be typically effective to perform the synthesis indicated. However, the person skilled in the art will appreciate that it may be necessary to vary the procedures for any given embodiment of the invention, e.g., vary the order or steps and/or the chemical reagents used. Products may be purified by conventional techniques that will vary, for example, according to the amount of side products produced and the physical properties of the compounds.

In the following examples, the compounds of formula (1) and the compounds of formula (2) used as starting materials may be prepared in accordance with WO2015/014722, which is incorporated by reference herein.

### EXAMPLE 1

### Synthetic preparation of Nicotinamide mononucleotide (NMN): Compound of Formula (I): R¹=hydrogen, n=0, Z²=NH, R²-R⁵=hydrogen, Y¹=sodium, Y²=internal salt with pyridinium

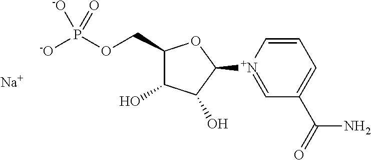

### Nicotinamide Mononucleotide (NMN)

To a dry 35 mL PTFE milling vessel containing one PTFE ball (0.8 cm diameter) was added nicotinamide riboside chloride (2000 mg, 6.88 mmol, 1.0 eq) and POCl₃ (2.57 mL, 27.52 mmol, 4.0 eq). The reaction was then milled at 30 Hz for 60 minutes or until the reaction had reached ˜95% conversion via c-18 HPLC analysis. The gum-like, gum-coated ball was removed and placed into a wide-necked flask, and the residue was solubilized in a minimal volume of distilled water over ice. The solution was adjusted to pH 6.0 by the drop-wise addition of a 2M NaOH solution. The aqueous solution was then reduced to a small volume under high vacuum and the pH was then adjusted to pH 3.0 using dilute nitric acid. To the aqueous solution was then added acetone (ca. 300 mL) and the precipitated white solid was separated and the supernatant discarded. The mixture was solubilized in a minimal quantity of distilled water and was purified on a 400 g C-18 snap cartridge at a flow rate of 50 mL/min using Biotage column chromatography (100% H₂O). The purified fractions were then freeze-dried to yield the pure product in 23% isolated yields as the monosodium salt.

¹H NMR (400 MHz, D₂O) δ ppm 9.32 (1H, s, Ar), 9.13 (1H, app. d, Ar), 8.89 (1H, dt, J=8.0, 1.3 Hz, Ar), 8.19 (1H, dd, J =8.0, 6.5 Hz, Ar), 6.04 (1H, d, J=5.5 Hz, H-1), 4.54 (1H, m, H-2), 4.46 (1H, t, J=5.1 Hz, H-3), 4.34 (1H, dd, J=5.0, 2.5 Hz, H-4), 4.21 (1H, ABX, J_{a,a}=12.0, 4.0 Hz, H-5), 4.05 (1H, ABX, J_{a,b}=12.0, 4.0 Hz, H-5). ¹³C NMR (75 MHz, D₂O) δ ppm 165.6 (C(═O)NH₂), 146.0, 142.5, 139.9, 133.9, 128.5, 99.9 (C-1, anomeric), 89.4 (C-4), 77.7 (C-2), 70.9 (C-3), 64.1 (C-5). ³¹P NMR (162 MHz, D₂O) δ ppm 0.03. HRMS (ES, M+H⁺) calculated for C₁₁H₁₄N₂O₈P 357.0488, found 357.047.

RP-HPLC Conditions for Example 1:

1) Preparation of 0.1M phosphate buffer pH 6.0:

i) 0.5 L of 1M K₂HPO₄ at 174.18 g.mol⁻¹=87.09 g

0.5 L of 1M KH₂PO₄ at 136.09 g.mol⁻¹=68.05 g

ii) 13.2 mL of 1M K₂HPO₄ and 86.8 mL of 1M KH₂PO₄ buffer diluted to 1 liter

Eluent A [0.1 M KH₂PO₄ buffer (pH 6)] and eluent B [0.1 M KH₂PO₄ buffer (pH 6) and 20% methanol] ran at a ratio of 75:25 AB over 20 min.

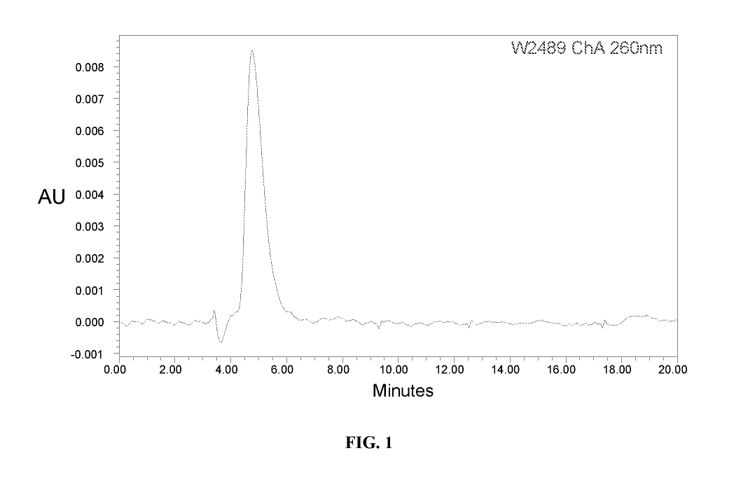

The HPLC chromatogram of Example 1 demonstrates the isolation of pure NMN (FIG. 1).

### Method 2 (Scale-up)

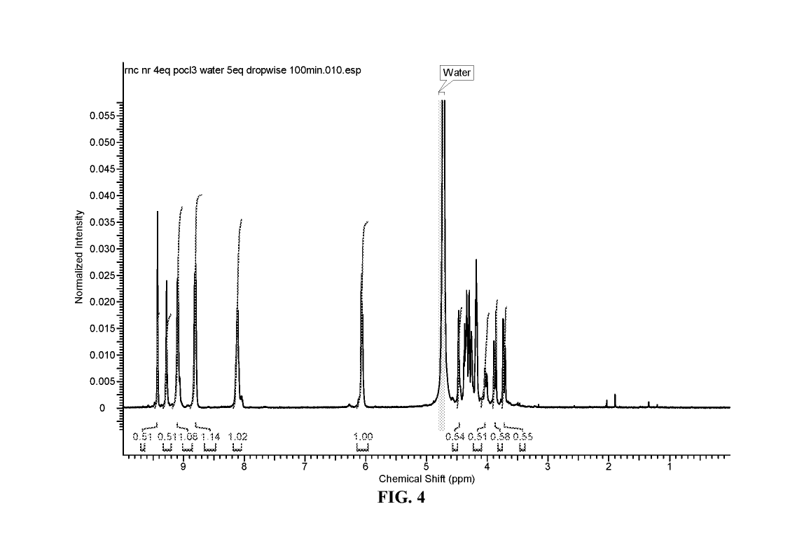

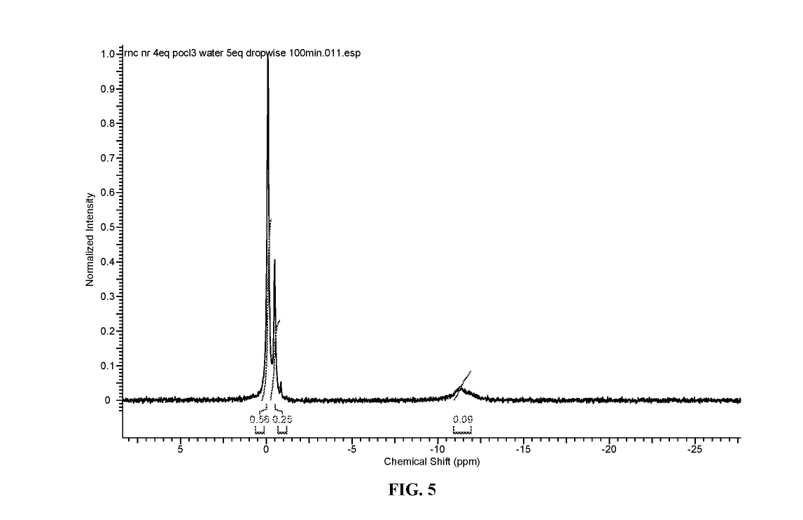

To a ceramic mortar was added NR chloride (40000 mg, 137.6 mmol, 1.0 eq) and POCl₃ (50.38 ml, 550.4 mmol, 4.0 eq), and this was then placed into an automated overhead grinder operated at 130 RPM. To this mixture, while stirring, was added water (12394.07 mg, 687.99 mmol, 5.0 eq), and then the mixture was mixed for a total of 60 minutes. Mixing was then continued for an additional 40 minutes. The off-white gummy mixture was then added slowly to ice water and the pH was adjusted to 6.0 by a saturated solution of NaHCO₃. The solution was then concentrated under high vacuum (or lyophilization), followed by purification on Dowex 1×2 formate type resin, using an eluent of pure water, and fractions containing the desired compound were combined and concentrated. Fractions containing unreacted NR were reisolated and could be recycled. The resin can then be regenerated using an eluent of 4M Formic acid. A subsequent column using Dowex 50W X8 yielded the desired NMN product as the inner salt. The reaction was quenched at 55% conversion; see the ¹H and ¹³P NMR spectra depicted in FIGS. 4 and 5, respectively.

### Method 3 (Assisted Grinding with Sulfolane)

To a ceramic mortar was added NR chloride (20000 mg, 68.8 mmol), POCl₃ (12.6 mL, 137.6 mmol), and sulfolane (8267.69 mg, 68.8 mmol, 1.0 eq), and this mixture was then placed into an automated overhead grinder operated at 130 RPM for 60 minutes. ¹H NMR analysis showed 30% conversion to the desired product.

### EXAMPLE 2

### Synthetic Preparation of Thiaminyl Monophosphate.

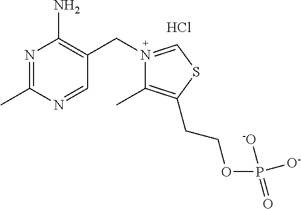

### Thiaminyl Monophosphate

To a dry 35 mL PTFE milling vessel containing one PTFE ball (0.8 cm diameter) was added thiamine HCl (2000 mg, 6.63 mmol, 1.0 eq) and POCl₃ (2.43 mL, 26.51 mmol, 4.0 eq). The reaction was then milled at 30 Hz for 60 minutes, or until the reaction had reached near completion by ¹H-NMR analysis. The white, paste-like residue was then solubilized in a minimal volume of distilled water over an ice bath and then concentrated, to give a fluffy white powder (92% conversion).

¹H NMR (400 MHz, D₂O) δ ppm 9.51 (s, 1H, Ar), 7.79 (s, 1H, Ar), 5.38 (s, 2H), 4.46 (q, J=5.4 Hz, 2H), 3.15 (t, J=4.9 Hz, 2H), 2.43 (s, 3H), 2.36 (s, 3H). ¹³C NMR (125 MHz, D₂O) δ ppm 163.2, 163.0, 155.0, 154.9, 144.3, 143.3, 135.5, 106.2, 64.8, 49.9, 27.5 (d, J=10.3 Hz), 20.9, 11.1. ³¹P NMR (162 MHz, D₂O) δ ppm −0.62.

The chlorination product is identified by chemical shifts observed at 2.99, 3.69, 7.83, and 9.47 ppm on the ¹H NMR spectrum. This chlorinated side product (i.e., chloro in place of phosphate ester group) can be minimized with the use of an overhead open-grinder, as per the above description.

### EXAMPLE 3

Synthetic Preparation of Pyridoxyl Monophosphate

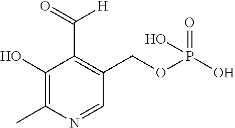 [SMILES: Cc1ncc(COP(=O)(O)O)c(C=O)c1O]

### Pyridoxyl Monophosphate

Pyridoxine (500 mg, 2.99 mmol, 1.0 eq) and POCl₃ (1.12 ml, 11.96 mmol, 4.0 eq) were added to a 50 mL ceramic mortar, and the mixture was then hand-grinded for 30 min total, using a ceramic pestle. ¹H NMR analysis showed 20% conversion to the desired product. ¹H NMR (400 MHz, D₂O) δ ppm 8.01 (1H, s, Aldehyde), 6.84 (1H, s, Ar), 5.24 (1H, m, CH₂O), 5.10 (1H, m, CH₂O), 2.49 (3H, s, CH₃). ³¹P NMR (162 MHz, D₂O) δ ppm −1.03.

### EXAMPLE 4

Synthetic Preparation of Nicotinic Acid Riboside Mononucleotide

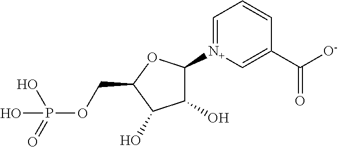 [SMILES: O=C([O-])c1ccc[n+]([C@@H]2O[C@H](COP(=O)(O)O)[C@@H](O)[C@H]2O)c1]

### Nicotinic Acid Riboside Mononucleotide

Nicotinic acid riboside (“NAR”) (2000 mg, 7.77 mmol, 1.0 eq) and POCl₃ (2.91 ml, 31.1 mmol, 4.0 eq) were added a ceramic mortar and was then hand-grinded for 30 min in total using a ceramic pestle. The product was isolated with 17% yield. ¹H NMR (400 MHz, D₂O) δ ppm 9.26 (1H, s, Ar), 9.13 (1H, d, J=6.3 Hz, Ar), 8.87-8.84 (1H, m, Ar), 8.12-8.09 (1H, m, Ar), 6.06 (1H, d, J=5.3 Hz, βH-1), 4.47 (1H, m, H-2), 4.40-4.37 (1H, m, H-3), 4.29-4.23 (1H, m, 2.5 Hz, 1H), 4.15 (1H, ABX, J_{a,a}=11.0, 4.0 Hz, H-5), 4.00 (1H, ABX, J_{a,a}=11.0, 4.0 Hz, 1H, H-5). ³¹P NMR (162 MHz, D₂O) δ ppm −0.25.

### EXAMPLE 5

Synthetic Preparation of Adenosyl Monophosphate

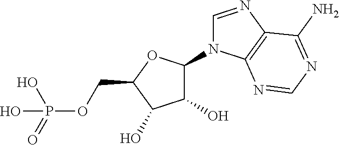 [SMILES: Nc1ncnc2c1ncn2[C@@H]1O[C@H](COP(=O)(O)O)[C@@H](O)[C@H]1O]

### Adenosyl Monophosphate

Adenosine (1000 mg, 3.74 mmol, 1.0 eq) was added to a ceramic mortar and POCl₃ (1.4 ml, 14.97 mmol, 4.0 eq) was added, and the mixture was hand-grinded using a ceramic pestle for 30 minutes. ¹H NMR analysis showed 15% conversion to the desired product.

### Method 2 (Water-assisted Grinding)

Adenosine (1000 mg, 3.74 mmol, 1.0 eq) was added to a ceramic mortar and phosphoryl trichloride (1.4 ml, 14.97 mmol, 4.0 eq), followed by 2 eq of water, was added and the mixture hand-grinded using a ceramic pestle for 30 minutes. ¹H NMR analysis showed 40% conversion to the desired product. C18 biotage chromatography using an eluent of 100% water yielded the desired product in 27% isolated yields and recovery of the unreacted adenosine. ¹H NMR (400 MHz, D₂O) δ ppm 8.50 (1H, s, Ar), 8.13 (1H, s, Ar), 6.01 (1H, m, J=6.0 Hz), 4.42-4.37 (1H, m), 4.28-4.22 (1H, m), 3.87 (2H, t, J=3.5 Hz). ³¹P NMR (162 MHz, D₂O) δ ppm 3.78.

### EXAMPLE 6

Reaction of NRH and P(O)(OEt)₂Cl: Compound of Formula (II): R¹=hydrogen, n=0, Z²=NH, R²-R⁷=hydrogen, Y¹=Y²=ethyl.

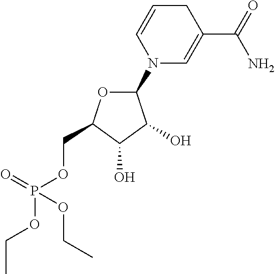 [SMILES: CCOP(=O)(OCC)OC[C@H]1O[C@@H](N2C=CCC(C(N)=O)=C2)[C@H](O)[C@@H]1O]

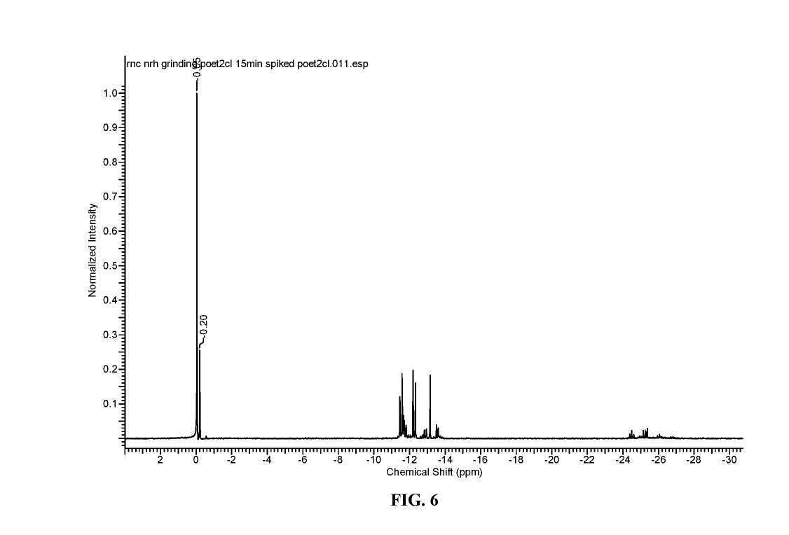

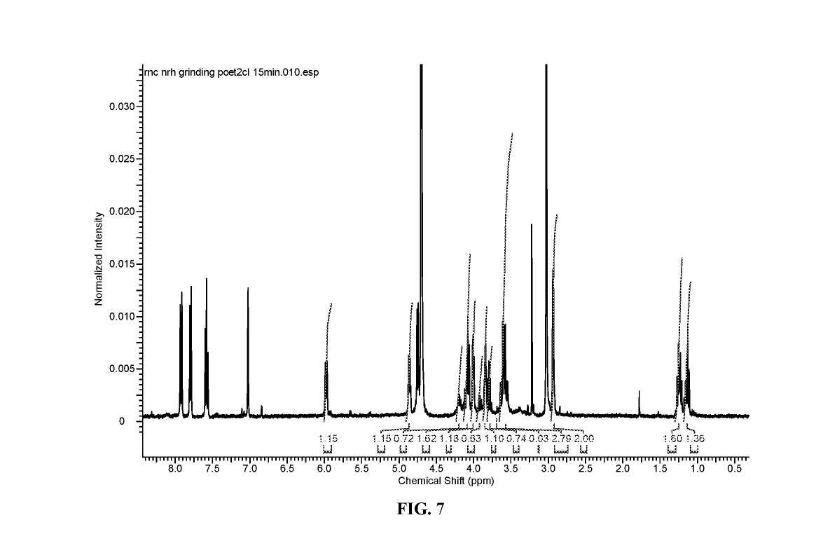

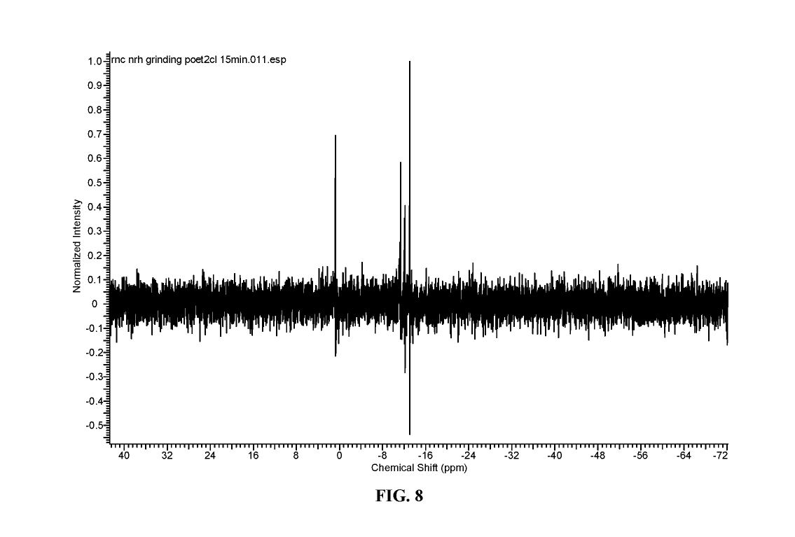

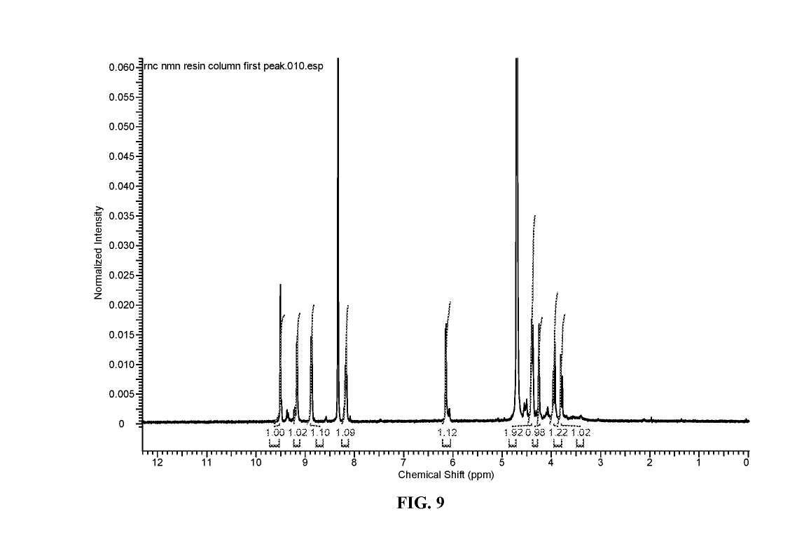

To a ceramic pestle and mortar was added dried NRH (200 mg, 0.78 mmol, 1.0 eq), ClPO(OEt)₂ (0.16 mL, 1.56 mmol, 2.0 eq) and proton sponge (334.04 mg, 1.56 mmol, 2.0 eq) the mixture was hand-ground using a ceramic pestle for 15 minutes. In FIG. 6, a ³¹P NMR spectrum shows a peak at −0.2 ppm for the phosphorylated reduced nucleoside, confirmed following spiking with excess ClPO(OEt)₂ and mixed for 5 minutes prior to phosphorus NMR analyses. Additionally, in FIG. 7, the ¹H NMR spectrum of the crude mixture indicates a shift in the riboside protons, in particular that of the C5 protons. FIG. 8 depicts a ¹³C NMR of the reaction product. FIG. 9 depicts a ¹H NMR spectrum demonstrating NR reagent recovered after reaction, with a minute quantity of NMN present. The NR reagent can be subsequently recycled.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method of making a compound of formula (I), or a salt thereof: 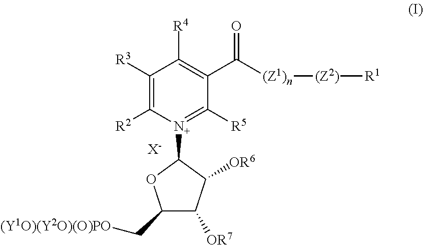
wherein X⁻ as counterion is absent, or when X⁻ is present is selected from the group consisting of fluoride, chloride, bromide, iodide, formate, acetate, ascorbate, benzoate, carbonate, citrate, carbamate, formate, gluconate, lactate, methyl bromide, methyl sulfate, nitrate, phosphate, diphosphate, succinate, sulfate, trifluoromethanesulfonate, and trifluoroacetate;
Y^{l} and Y² are independently selected from the group consisting of hydrogen, sodium, potassium, lithium, substituted or unsubstituted (C₁-C₈)alkyl, substituted or unsubstituted (C₁-C₈)cycloalkyl, substituted or unsubstituted aryl, substituted or unsubstituted heteroaryl, substituted or unsubstituted heterocycle, andsubstituted or unsubstituted amino; or alternatively, Y¹ and Y² taken together are selected from the group consisting of sodium, potassium, lithium, magnesium, calcium, strontium, and barium;
Z¹ and Z² are independently NH or oxygen;
n is 0 or 1;
R¹ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of (C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, NR^{C}C(O)R^{C}, NR^{C}C(O)O(C₁-C₆)alkyl, NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R^{A} is selected from the group consisting of —H, —(C₁-C₆)alkyl, —(CH₂)₃—NH—C(NH₂)(═NH), —CH₂C(═O)NH₂, —CH₂COOH, —CH₂SH, —(CH₂)₂C(═O)—NH₂, —(CH₂)₂COOH, —CH₂-(2-imidazolyl), —CH(CH₃)—CH₂—CH₃, —CH₂CH(CH₃)₂, —(CH₂)₄—NH₂, —(CH₂)₂—S—CH₃, phenyl, —CH₂-phenyl, —CH₂—OH, —CH(OH)—CH₃, —CH₂-(3-indolyl), —CH₂-(4-hydroxyphenyl), —CH(CH₃)₂, and —CH₂—CH₃;
R^{B} is hydrogen or —(C₁-C₈)alkyl;
each R^{c} is independently selected from the group consisting of hydrogen and (C₁-C₈)alkyl; and
R², R³, R⁴, and R⁵ are each independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R⁶ and R⁷ are independently selected from the group consisting of hydrogen, —C(O)R′, —C(O)OR′, —C(O)NHR′, substituted or unsubstituted (C₁-C₈)alkyl, substituted or unsubstituted (C₁-C₈)cycloalkyl, substituted or unsubstituted aryl, substituted or unsubstituted heteroaryl, substituted or unsubstituted heterocycle, substituted or unsubstituted aryl(C₁-C₄)alkyl, and substituted or unsubstituted heterocycle(C₁-C₄)alkyl; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R′ is selected from the group consisting of hydrogen, —(C₁-C₈)alkyl, —(C₁-C₈)cycloalkyl, aryl, heteroaryl, heterocycle, aryl(C₁-C₄)alkyl, and heterocycle(C₁-C₄)alkyl; and
R″ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, (C₁-C₆)perfluoroalkyl, and (C₁-C₆)alkyleneOR^{C};
provided that when Z² is NH, the absolute configuration of C* is D or L, or a mixture of D and L;
comprising the steps of:
(a) providing a compound or derivative having formula (1), or a salt thereof: 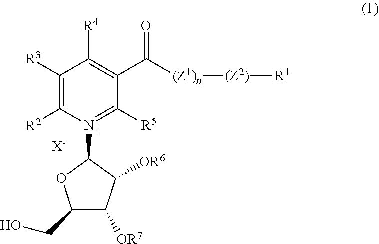
wherein X⁻ as counterion is absent, or when X⁻ is present is selected from the group consisting of fluoride, chloride, bromide, iodide, formate, acetate, ascorbate, benzoate, carbonate, citrate, carbamate, formate, gluconate, lactate, methyl bromide, methyl sulfate, nitrate, phosphate, diphosphate, succinate, sulfate, trifluoromethanesulfonate, and trifluoroacetate;
Z¹ and Z² are independently NH or oxygen;
n is 0 or 1;
R¹ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R^{A} is selected from the group consisting of —H, —(C₁-C₆)alkyl, —(CH₂)₃—NH—C(NH₂)(═NH), —CH₂C(═O)NH₂, —CH₂COOH, —CH₂SH, —(CH₂)₂C(—O)—NH₂, —(CH₂)₂COOH, —CH₂-(2-imidazolyl), —CH(CH₃)—CH₂—CH₃, —CH₂CH(CH₃)₂, —(CH₂)₄—NH₂, —(CH₂)₂—S—CH₃, phenyl, —CH₂-phenyl, —CH₂—OH, —CH(OH)—CH₃, —CH₂-(3-indolyl), —CH₂-(4-hydroxyphenyl), —CH(CH₃)₂, and —CH₂—CH₃;
R^{B} is hydrogen or —(C₁-C₈)alkyl;
each R^{C} is independently selected from the group consisting of hydrogen and —(C₁-C₈)alkyl;
R², R³, R⁴, and R⁵ are each independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R⁶ and R⁷ are independently selected from the group consisting of hydrogen, —C(O)R′, —C(O)OR′, —C(O)NHR′, substituted or unsubstituted (C₁-C₈)alkyl, substituted or unsubstituted (C₁-C₈)cycloalkyl, substituted or unsubstituted aryl, substituted or unsubstituted heteroaryl, substituted or unsubstituted heterocycle, substituted or unsubstituted aryl(C₁-C₄)alkyl, and substituted or unsubstituted heterocycle(C₁-C₄)alkyl, wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R′ is selected from the group consisting of hydrogen, —(C₁-C₈)alkyl, —(C₁-C₈)cycloalkyl, aryl, heteroaryl, heterocycle, aryl(C₁-C₄)alkyl, and heterocycle(C₁-C₄)alkyl;
R″ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkyl ene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
provided that when Z² is NH, the absolute configuration of C* is D or L, or a mixture of D and L;
(b) treating the compound of formula (1) with phosphorus oxychloride;
(c) mechanically processing the components;
(d) adding water to the mixture;
(e) adjusting the pH with an aqueous base; and
precipitating the compound of formula (I).

2. The method of claim 1, further comprising step:
(g) purifying and/or isolating the compound of formula (I).

3. The method of claim 2, wherein the compound of formula (I) is freeze dried.

4. The method of claim 1, wherein the mechanically processing step comprises one or more methods of agitation selected from the group consisting of grinding, mixing, milling, triturating, and liquid-assisted milling.

5. The method of claim 4, wherein the mixing and/or milling is performed between about 20 Hz and about 30 Hz for about 60 min to about 120 min.

6. The method of claim 4, wherein the grinding is performed between about 100 RPM and about 130 RPM.

7. A method of making a compound of formula (II), or a salt thereof: 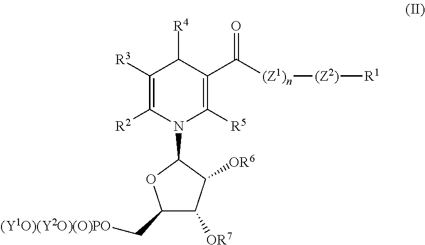
wherein Y¹ and Y² are independently selected from the group consisting of hydrogen, sodium, potassium, lithium, substituted or unsubstituted (C₁-C₈)alkyl, substituted or unsubstituted (C₁-C₈)cycloalkyl, substituted or unsubstituted aryl, substituted or unsubstituted heteroaryl, substituted or unsubstituted heterocycle, and substituted or unsubstituted amino; or alternatively, Y¹ and Y² taken together are selected from the group consisting of sodium, potassium, lithium, magnesium, calcium, strontium, and barium;
Z¹ and Z² are independently NH or oxygen;
n is 0 or 1;
R¹ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R^{A} is selected from the group consisting of —H, —(C₁-C₆)alkyl, —(CH₂)₃—NH—C(NH₂)(═NH), —CH₂C(═O)NH₂, —CH₂COOH, —CH₂SH, —(CH₂)₂C(═O)—NH₂, —(CH₂)₂COOH, —CH₂-(2-imidazolyl), —CH(CH₃)—CH₂—CH₃, —CH₂CH(CH₃)₂, —(CH₂)₄—NH₂, —(CH₂)₂—S—CH₃, phenyl, —CH₂-phenyl, —CH₂—OH, —CH(OH)—CH₃, —CH₂-(3-indolyl), —CH₂-(4-hydroxyphenyl), —CH(CH₃)₂, and —CH₂—CH₃;
R^{B} is hydrogen or —(C₁-C₈)alkyl;
each R^{C} is independently selected from the group consisting of hydrogen and —(C₁-C₈)alkyl; and
R², R³, R⁴, and R⁵ are each independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C6)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R⁶ and R⁷ are independently selected from the group consisting of hydrogen, —C(O)R′, —C(O)OR′, —C(O)NHR′, substituted or unsubstituted (C₁-C₈)alkyl, substituted or unsubstituted (C₁-C₈)cycloalkyl, substituted or unsubstituted aryl, substituted or unsubstituted heteroaryl, substituted or unsubstituted heterocycle, substituted or unsubstituted aryl(C₁-C₄)alkyl, and substituted or unsubstituted heterocycle(C₁-C₄)alkyl; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R′ is selected from the group consisting of hydrogen, —(C₁-C₈)alkyl, —(C₁-C₈)cycloalkyl, aryl, heteroaryl, heterocycle, aryl(C₁-C₄)alkyl, and heterocycle(C₁-C₄)alkyl;
R″ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
provided that when Z² is NH, the absolute configuration of C* is D or L, or a mixture of D and L;
comprising the steps of:
(a) providing a compound or derivative having formula (2), or a salt thereof: 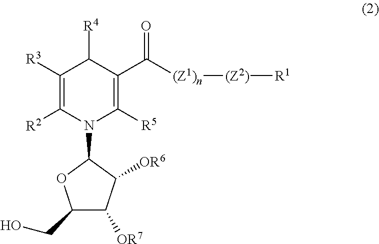
wherein Z¹ and Z² are independently NH or oxygen;
n is 0 or 1;
R¹ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R^{A} is selected from the group consisting of —H, —(C₁-C₆)alkyl, —(CH₂)₃-NH—C(NH₂)(═NH), —CH₂C(═O)NH₂, —CH₂COOH, —CH₂SH, —(CH₂)₂C(═O)—NH₂, —(CH₂)₂COOH, —CH₂-(2-imidazolyl), —CH(CH₃)—CH₂—CH₃, —CH₂CH(CH₃)₂, —(CH₂)₄—NH₂, —(CH₂)₂—S—CH₃, phenyl, —CH₂-phenyl, —CH₂—OH, —CH(OH)—CH₃, —CH₂-(3-indolyl), —CH₂-(4-hydroxyphenyl), —CH(CH₃)₂, and —CH₂—CH₃;
R^{B} is hydrogen or —(C₁-C₈)alkyl;
each R^{C} is independently selected from the group consisting of hydrogen and —(C₁-C₈)alkyl; and
R², R³, R⁴, and R⁵ are each independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R⁶ and R⁷ are independently selected from the group consisting of hydrogen, —C(O)R′, —C(O)OR′, —C(O)NHR′, substituted or unsubstituted (C₁-C₈)alkyl, substituted or unsubstituted (C₁-C₈)cycloalkyl, substituted or unsubstituted aryl, substituted or unsubstituted heteroaryl, substituted or unsubstituted heterocycle, substituted or unsubstituted aryl(C₁-C₄)alkyl, and substituted or unsubstituted heterocycle(C₁-C₄)alkyl; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
R′ is selected from the group consisting of hydrogen, —(C₁-C₈)alkyl, —(C₁-C₈)cycloalkyl, aryl, heteroaryl, heterocycle, aryl(C₁-C₄)alkyl, and heterocycle(C₁-C₄)alkyl;
R″ is selected from the group consisting of hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin B1 ester, vitamin B2 ester, vitamin B6 ester, choline ester, biotin ester, vitamin A ester, resveratrol ester, and —C*H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group consisting of —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NR^{C}C(O)R^{C}, —NR^{C}C(O)O(C₁-C₆)alkyl, —NR^{C}C(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};
provided that when Z² is NH, the absolute configuration of C* is D or L, or a mixture of D and L;
(b) treating the compound of formula (2) with a base;
(c) mechanically processing the components in the presence of phosphorus oxychloride;
(d) adding water to the mixture;
(e) filtering;
(f) adjusting the pH with an aqueous base; and
(g) precipitating the compound of formula (II).

8. The method of claim 7, further comprising step:
(h) purifying and/or isolating the compound of formula (II).

9. The method of claim 8, wherein the compound or formula (II) is freeze-dried.

10. The method of claim 7, wherein the mechanically processing step comprises one or more methods of agitation selected from the group consisting of grinding, mixing, milling, trituration, and liquid-assisted milling.

11. The method of claim 10, wherein the mixing and/or milling is performed between about 20 Hz and 30 Hz for about 60 min to about 120 min.

12. The method of claim 10, wherein the grinding is performed between about 100 RPM and about 130 RPM.

13. A method of making thiaminyl monophosphate, or a salt thereof, comprising the steps of:
(a) providing thiamine or a salt thereof;
(b) treating the thiamine or salt thereof with phosphorus oxychloride;
(c) mechanically processing the components;
(d) adding water to the mixture;
(e) adjusting the pH with an aqueous base; and
precipitating the thiaminyl monophosphate.

14. The method of claim 13, further comprising step:
(g) purifying and/or isolating the thiaminyl monophosphate.

15. The method of claim 14, wherein the thiaminyl monophosphate is freeze dried.

16. The method of claim 13, wherein the mechanically processing step comprises one or more methods of agitation selected from the group consisting of grinding, mixing, milling, trituration, and liquid-assisted milling.

17. The method of claim 16, wherein the mixing and/or milling is performed between about 20 Hz and about 30 Hz for about 60 min to about 120 min.

18. The method of claim 16, wherein the grinding is performed between about 100 RPM and about 130 RPM.

19. A method of making pyridoxyl monophosphate, or a salt thereof, comprising the steps of:
(a) providing pyridoxine or a salt thereof;
(b) treating the pyridoxine or salt thereof with phosphorus oxychloride;
(c) mechanically processing the components;
(d) adding water to the mixture;
(e) adjusting the pH with an aqueous base; and
(f) precipitating the pyridoxyl monophosphate.

20. The method of claim 19, further comprising step:
(g) purifying and/or isolating the pyridoxyl monophosphate.

21. The method of claim 20, wherein the pyridoxyl monophosphate is freeze dried.

22. The method of claim 19, wherein the mechanically processing step comprises one or more methods of agitation selected from the group consisting of grinding, mixing, milling, trituration, and liquid-assisted milling.

23. The method of claim 22, wherein the mixing and/or milling is performed between about 20 Hz and about 30 Hz for about 60 min to about 120 min.

24. The method of claim 22, wherein the grinding is performed between about 100 RPM and about 130 RPM.

25. A method of making adenosyl monophosphate, or a salt thereof, comprising the steps of:
(a) providing adenosine or a salt thereof;
(b) treating the adenosine or salt thereof with phosphorus oxychloride;
(c) mechanically processing the components;
(d) adding water to the mixture;
(e) adjusting the pH with an aqueous base; and
precipitating the adenosyl monophosphate.

26. The method of claim 25, further comprising step:
(g) purifying and/or isolating the adenosyl monophosphate.

27. The method of claim 26, wherein the adenosyl monophosphate is freeze dried.

28. The method of claim 25, wherein the mechanically processing step comprises one or more methods of agitation selected from the group consisting of grinding, mixing, milling, trituration, and liquid-assisted milling.

29. The method of claim 28, wherein the mixing and/or milling is performed between about 20 Hz and about 30 Hz for about 60 min to about 120 min.

30. The method of claim 28, wherein the grinding is performed between about 100 RPM and about 130 RPM.
